(19) United States
(12) Patent Application Publication
(10) Pub. No.: US 2024/0344056 A1
(43) Pub. Date: Oct. 17, 2024

(54) IMPROVED METHOD OF SEQUENCING LIBRARY PREPARATION FOR SHORT DNA

(71) Applicant: SomaGenics, Inc., Santa Cruz, CA (US)

(72) Inventors: Sergei A. Kazakov, Santa Cruz, CA (US); Ryan E. Hogans, Santa Cruz, CA (US); Colin Hortman, Santa Cruz, CA (US); Yolanda F.A. Townsley, Santa Cruz, CA (US); Rachel Harbeitner, Santa Cruz, CA (US); Brian H. Johnston, Santa Cruz, CA (US)

(21) Appl. No.: 18/566,839

(22) PCT Filed: Jun. 2, 2022
(86) PCT No.: PCT/US2022/032008

§ 371 Date: May 28, 2024

Related U.S. Application Data
(60) Provisional application No. 63/197,169, filed on Jun. 4, 2021.

(51) Int. Cl. C12N 15/10; C12N 15/11

(52) U.S. Cl. CPC C12N 15/1093; C12N 15/11; C12N 2310/531

(57) ABSTRACT

Disclosed herein are compositions and methods for the detection of DNAs in a sample, including single-stranded DNA, denatured double-stranded DNAs and DNA fragments for research and clinical diagnostic purposes. The methods and compositions disclosed herein may be used for preparing next generation sequencing libraries of highly fragmented DNA molecules isolated from biofluids (such as plasma, serum, saliva and urine), FFPE samples and ancient organisms. The methods involve the ligation of hairpin adaptors to the 3′-end Sand the 5′-end of the ssDNA.

### CROSS-REFERENCE

This application claims the benefit of U.S. Provisional Application No. 63/197,169, filed on Jun. 4, 2021, which is incorporated herein by reference in its entirety.

### GOVERNMENT SUPPORT

This invention was made with government support under Small Business Innovation Research grant 2R44HG009461-02A1 (5R44HG009461-03) awarded by the National Institute of Health. The government has certain rights in the invention.

### SUMMARY

In certain aspects, described herein is a composition suitable for preparation of a single-stranded DNA (ssDNA) sequencing library comprising: (a) a first hairpin adapter (HPA1) and a second hairpin adapter (HPA2) comprised of a plurality of residues selected from: nucleic acid residues, modified nucleic acid residues, non-nucleotide residues or a combination thereof, wherein the HPA1 and the HPA2 comprise: i) a first segment comprising at least one primer-specific or probe-specific sequence; ii) a second segment comprising a sequence substantially complementary to the first segment; and iii) a loop connecting the first segment and the second segment; wherein a free end of the second segment comprises an overhang of at least one residue, wherein the second segment comprises a cleavable moiety located at least 9 nucleotides distance from the free end of the second segment; wherein a free end of the first segment of HPA1 is capable of ligating to a first end of a sample ssDNA, and a free end of the first segment of HPA2 is capable of ligating to a second end of a sample ssDNA. In some embodiments, the HPA1 and the HPA2 consist of: i) a first segment comprising at least one primer-specific or probe-specific sequence; ii) a second segment comprising a sequence substantially complementary to the first segment; and iii) a loop connecting the first segment and the second segment; wherein a free end of the second segment comprises an overhang of at least one residue, wherein the second segment comprises a cleavable moiety located at least 9 nucleotides distance from the free end of the second segment; wherein a free end of the first segment of HPA1 is capable of ligating to a first end of a sample ssDNA, and a free end of the first segment of HPA2 is capable of ligating to the other segment of the sample ssDNA. In some embodiments, the free end of the second segment of the HPA1, the free end of the second segment of the HPA2, or the free end of the second segment of the HPA1 and the free end of the second segment of the HPA2 comprise a blocking group that prevents its ligation by a ligase and/or extension by a polymerase. In some embodiments, the primer-specific and/or probe-specific sites are at least partially complementary or corresponding to sequences of primers and probes selected from: amplification primers, sequencing primers, detection primers, detection probes, hybridization probes, anchor probes, linker probes, capture probes or combination thereof. In some embodiments, the first segment and the second segment form a double-stranded stem structure with at least 1, 2, 3, or more mismatches. In some embodiments, the double-stranded stem structure comprises 3 or more nucleotide base pairs. In some embodiments, the loop comprises 1 or more residues selected from: a nucleotide, a modified nucleotide and a non-nucleotide linker or moiety. In some embodiments, the cleavable residue or moiety is selected from: a RNA, a deoxyuridine (dU), a deoxyinosine (dI); an internucleotide disulfide (S—S) linker and an internucleotide, bridging or non-bridging phosphorothioate (PS) linkage. In some embodiments, the overhang comprises three or more randomized nucleotide residues (N) or defined nucleic acid and/or modified nucleic acid residues or combinations thereof, allowing simultaneous ligation with any sample ssDNA regardless of its end sequences. In some embodiments, the ligation is target-specific. In some embodiments, the adaptor overhang comprises a sequence that is substantially complementary to a sequence of a sample ssDNA end. In some embodiments, the overhang comprises from 3 to 12 randomized residues selected from: nucleotide residues, modified nucleotide residues, or a combination thereof. In some embodiments, the modified nucleotide residues are selected from the list consisting of Locked nucleic acids, 2′-OMethyl, 2′-Fluoro, 2-Amino-dA, 5-Methyl-dC, C-5 propynyl-C, and C-5 propynyl-U. In some embodiments, the free end of HPA1 is selected from: 5′-hydroxyl (5′-OH), 5′-phosphate (5′-p), 3′-hydroxyl (3′-OH), 3′-phosphate (3′-p) or combination thereof. In some embodiments, the free end of HPA2 is selected from: 5′-OH, 5′-p, 3′-OH, 3′-p or combination thereof. In some embodiments, the HPA1 or the HPA2 is a 3′-HPA that can be ligated to the 3′-end of a sample ssDNA. In some embodiments, the HPA1 or the HPA2 is a 5′-HPA that can be ligated to the 5′-end of a sample ssDNA. In some embodiments, the composition further comprises a blocking oligonucleotide (BON) comprising: i) a free end that enables ligation of the BON with any HPA1 remaining unligated after the the ligation of the HPA1 to the ssDNA to reduce ligation between the HPA1 and the HPA2; and ii) a second end. In some embodiments, the second end of the BON comprises a blocking group that disallows its ligation and/or extension by a polymerase. In some embodiments, the BON comprises a structure selected from: single-stranded, double-stranded, hairpin, or combination thereof. In some embodiments, the BON comprises a nucleotide selected from: DNA, RNA; a randomized nucleotide residue (N); a defined nucleic acid residue; a modified nucleic acid residue; or a combination thereof. In some embodiments, the BON comprises from 1 to 12 randomized nucleotide residues. In some embodiments, the free end of the BON or the second end of the BON comprises sequences selected from 4 to 6 random nucleotides. In some embodiments, the free end of the BON or the second end of the BON are selected from the group consisting of: a 5′-OH, 5′-p, 3′-OH, and combinations thereof. In some embodiments, the blocking group is selected from: 5′-OH, 5′-amino, 5′-O-methyl and 5′-biotin linker (5′-end blocking groups); and 3′-p, dideoxynucleoside (e.g., 3′-ddC), 3-inverted dT (idT), 3′-C3 spacer, 3′-amino, and 3′-biotin linker (3′-end blocking groups).

In certain aspects, described herein is a method for preparing a plurality of primer extension products for a plurality of sample ssDNAs, comprising: a) ligating the HPA1 described herein to the first end of a plurality of sample ssDNAs to produce a mixture comprising a plurality of HPA1-ssDNA or ssDNA-HPA1 ligation products and unligated HPA1; b) ligating the HPA2 described herein to the second end of the HPA1-ssDNA ligation products to produce 5′-HPA-ssDNA-3′-HPA (5′-HPA1-ssDNA-HPA2-3′ or 5′-HPA2-ssDNA-HPA1-3′) ligation products, wherein the sample ssDNA is positioned between the HPA1 and HPA2; c) cleaving the of the HPA(s) to produce cleaved 5′-HPA-DNA-3′-HPA ligation products, wherein the cleaving converts the HPAs to opened forms that are more accessible for hybridization with primer(s) and/or probe(s); and d) hybridizing a first primer comprising a sequence at least partially complementary to the 3′-HPA(s) segment of the cleaved 5′-HPA-ssDNA-3′-HPA products; and extending the primer with a polymerase to produce a plurality of the first primer extension products. In some embodiments, the method further comprises (e) hybridizing a second primer comprising a sequence at least partially complementary to the 3′-end of the first primer extension products comprising a sequence complementary to the 5′-HPA segment, and extending the primer with a polymerase to produce a plurality of the second primer extension products comprising a sequencing library. In some embodiments, the ligating of the HPA1 in step (a) occurs before the ligating of HPA2 in step (b). In some embodiments, the ligating of the HPA1 in step (a) and the ligating of the HPA2 in step (b) occur simultaneously. In some embodiments, the method further comprises step (f) ligating any HPA1 remaining unligated after step (a) with the BON described herein to produce HPA1-BON ligation product(s) that prevent its ligation to HPA2 (adapter dimers formation) in a downstream ligation reaction. In some embodiments, the method further comprises removing components of upstream reactions that may inhibit downstream primer extension reactions. In some embodiments, the removing is performed using SPRIselect beads and reagents. In some embodiments, the method further comprises amplifying the sequencing library to produce an amplified sequencing library. In some embodiments, the method further comprises performing a post-amplification clean-up to purify the amplified sequencing library from non-extended primers and other components of extension and/or amplification reactions. In some embodiments, the sample ssDNAs are naturally occurring and/or synthetic DNA molecules selected from: single-stranded DNAs; fragmented single-stranded DNAs; denatured double-stranded DNAs; denatured fragmented double-stranded DNAs. In some embodiments, the sample ssDNAs are short ssDNA fragments of 120 or fewer nucleotides in length. In some embodiments, the sample ssDNAs are ultrashort ssDNA fragments in the range of 20 to 80 nucleotides in length. In some embodiments, the sample ssDNAs are in the range of 18 to 50 nucleotides in length. In some embodiments, the sample ssDNAs are selected from: circulating tumor DNA, circulating microbial DNA, circulating bacterial DNA, circulating viral DNA, circulating mitochondrial DNA, circulating genomic DNA, circulating cell-free DNA (cfDNA) from a biofluid (liquid biopsy); DNA from formalin-fixed, paraffin-embedded (FFPE) tissue samples; highly degraded DNA from ancient organisms or forensic biological samples. In some embodiments, the biofluid is selected from: whole blood, plasma, serum, saliva, and urine. In some embodiments, the sample ssDNA comprises isolated total nucleic acids including both DNA and RNA. In some embodiments, the sample ssDNA comprises isolated total DNA. In some embodiments, the sample ssDNA comprises isolated target DNAs. In some embodiments, the target ssDNAs are isolated by hybridization with target-specific oligonucleotide probes. In some embodiments, the hybridization is performed either in solution followed by attachment of target-probe complexes to a solid support, or on a solid phase comprising target-specific probes immobilized on a solid support. In some embodiments, the target ssDNAs are captured directly from a biofluid or a lysed biofluid. In some embodiments, the plurality of sample ssDNAs comprise DNA ends selected from 5′-p, 3′-OH, 5′-OH and 3′-p or combinations thereof. In some embodiments, the method further comprises chemically or enzymatically treating the sample ssDNAs. In some embodiments, chemically or enzymatically treating the sample ssDNAs comprises a bisulfite treatment protocol that can convert unmethylated cytosine (C) to deoxyuridine (dU). In some embodiments, chemically or enzymatically treating the sample ssDNAs comprises repairing the sample ssDNA to convert 5′-OH and/or 3′-p ends to 5′-p and 3′-OH forms. In some embodiments, the repairing is performed by a polynucleotide kinase. In some embodiments, the ligating is performed without repair of ends of the sample ssDNA. In some embodiments, the ssDNAs are chemically or enzymatically treated prior to the ligating of HPA1 in step (a) or the ligating of HPA2 in step (b). In some embodiments, the ssDNAs are chemically or enzymatically treated simultaneously with the ligating of HPA1 in step (a) or the ligating of HPA2 in step (b). In some embodiments, the HPA1 is a 3′-HPA and the HPA2 is a 5′-HPA. In some embodiments, the HPA1 is the 5′-HPA and the HPA2 is the 3′-HPA. In some embodiments, the 3′-HPA comprises a 5′-p end and its 3′-overhang comprises a 3′-end blocking group. In some embodiments, the 5′-HPA comprises a 3′-OH end and a 5′-end overhang comprising a 5′-OH or a 5′-end blocking group. In some embodiments, the remaining unligated 3′-HPA is blocked by ligating with a 3′-blocking oligonucleotide (3′-BON) comprising a 3′-OH and a 5′-OH or 5′-end-blocking group. In some embodiments, the remaining unligated 5′-HPA is blocked by ligating with a 5′-blocking oligonucleotide (5′-BON) comprising a 5′-p end and a 3′-end-blocking group. In some embodiments, the HPA2 is taken in molar excess over the HPA1. In some embodiments, the BONs are taken in molar excess over the HPA1 during the blocking step. In some embodiments, the ligating is splint-dependent ligation between the sample ssDNAs, the HPA1, the HPA2 and/or the BON and a ligation step performed by a ligase. In some embodiments, the ligase is selected from: Salt-T4® DNA Ligase, T4 DNA ligase, T3 DNA ligase, T7 DNA ligase, PBCV1 DNA ligase, E. coli DNA ligase, human DNA ligase III, and SplintR® Ligase. In some embodiments, the ligating by T4 DNA ligase is performed in the presence of ATP at a concentration from about 50 to about 100 μM. In some embodiments, the HPA1 or the HPA2 comprise one or more RNA residues that can be cleaved by an RNA cleaving agent selected from: a ribonuclease, a ribozyme, a deoxyribozyme, basic buffer solutions, alkaline solutions, divalent or multivalent metal cations, or combinations thereof. In some embodiments, the ribonuclease comprises RNase H. In some embodiments, the use of a RNA cleaving agent for the HPA cleavage simultaneously cleaves any RNA present in the sample ssDNA to prevent the incorporation of RNA sequences into the ssDNA sequencing library. In some embodiments, the RNA cleaving agent is using oligonucleotides comprising sequence-specific or randomized nucleotides to guide a cleavage of RNA molecules incorporated into the ssDNA sequencing library. In some embodiments, the amplification is performed by PCR using a thermostable DNA polymerase. In some embodiments, the thermostable DNA polymerase is selected from: LongAmp HotStart Taq DNA polymerase, KAPA HiFi HotStart DNA polymerase, KAPA HiFi HotStart Uracil+DNA polymerase; Pfu Turbo Cx HotStart DNA polymerase.

In certain aspects, described herein is a method for preparing a sequencing library from a plurality of sample ssDNAs, comprising: a) ligating a 5′-hairpin adapter (5′-HPA) to the 5′-ends of the plurality of sample single-stranded DNA (sample ssDNAs) to produce a mixture comprising a plurality of 5′-HPA-ssDNA ligation products, wherein the 5′-HPA is comprised of a plurality of residues selected from: nucleic acid residues (DNA and RNA), modified nucleic acid residues, non-nucleotide residues or a combination thereof, wherein the 5′-HPA comprises: i) a first segment comprising at least one primer primer-specific sequence; ii) a second segment comprising a sequence substantially complementary to the first segment, iii) a loop connecting the first segment and the second segment; iv) free 3′-end and 5′-end which are not connected to the loop; wherein the free 3′ end of the first segment comprises 3′-OH; and the 5′ end of the second segment comprises an overhang of 5 or 6 randomized nucleotide residues and 5′-OH; and wherein the second segment comprises 2 or more RNA residues in its stem region located at least 9 nucleotides distance from the free 5′ end of the second segment; and b) ligating the 3′-hairpin adapter (3′-HPA) to the 3′ ends of the 5′-HPA-ssDNA ligation products to produce 5′-HPA-ssDNA-3′-HPA ligation products; wherein the 3′-HPA is comprised of a plurality of residues selected from: nucleic acid residues (DNA and RNA), modified nucleic acid residues, non-nucleotide residues or a combination thereof, wherein the 3′-HPA comprises: i) a first segment comprising at least one primer primer-specific sequence; ii) a second segment comprising a sequence substantially complementary to the first segment, iii) a loop connecting the first segment and the second segment; iv) free 5′-end and 3′-end which are not connected to the loop; wherein the free 5′ end of the first segment comprises 5′-p; and 3′ end of the second segment comprises an overhang of 5 or 6 randomized nucleotide residues and 3′-blocking group that prevents its ligation and/or extension by a polymerase; and wherein the second segment comprises 2 or more RNA residues in its stem region; and c) cleaving the RNA residues in both 5′-HPAs and 3′-HPAs by treatment with an RNase H to produce cleaved 5′-HPA-ssDNA-3′-HPA ligation products, wherein the cleaving makes the first segments of 5′-HPA and 3′-HPA more accessible for hybridization with primers; d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification; e) amplifying the cleaved 5′-HPA-ssDNA-3′-HPA ligation products by a PCR using a pair of the first and the second sequencing primers to produce (amplified) sequencing library, wherein: i) the first sequencing primer is at least partially complementary at its 3′ end to the 3′-HPA segment of the cleaved 5′-HPA-ssDNA-3′-HPA ligation products; and ii) the second sequencing primer is at least partially corresponding at its 3′ end to the 5′-HPA segments of the cleaved 5′-HPA-ssDNA-3′-HPA ligation products; f) performing a post-amplification clean-up to purify the (amplified) sequencing library from non-extended primers and other components of extension and/or amplification reactions to produce purified sequencing libraries that are ready for sequencing. In some embodiments, the ligating of 3′-HPA (but not 5′-HPA) is performed in the presence of polynucleotide kinase (PNK). In some embodiments, the HPA1, the HPA2 and the sequencing primers are compatible with current high-throughput, sequencing technologies selected from: second-generation or next-generation sequencing (NGS) and third-generation or direct, single-molecule sequencing. In some embodiments, the sequencing is selected from sequencing methods provided by sequencing-by-synthesis, single-molecule real time sequencing, and nanopore sequencing. In some embodiments, the primers are sequencing primers comprising molecular codes selected from: bar-codes, sequencing indexes, unique molecular identifiers (UMI or UID) or combination thereof.

In certain aspects, described herein is a kit for preparing the sample ssDNA sequencing library comprising a 5′-HPA, a 3′-HPA, a ligase, an RNA cleaving agent, buffers, and optional components selected from: PNK, a polymerase, a 5′-BON, 3′-BON, and clean-up beads or combinations thereof.

### BRIEF DESCRIPTION OF THE DRAWINGS

FIG. 1A-1C. Schemes to exemplify Hairpin Adapters (HPAs). FIG. 1A: General structures of 5′-Hairpin Adapter (5′-HPA) and 3′-Hairpin Adapter (3′-HPA), each comprising a double-stranded stem region formed by complementary first and second segments, a loop connecting one end of the first and one end of the second segments, and a single-stranded overhang on the free end of the second segment. FIG. 1B: Examples of different HPA designs comprising DNA, cleavable segments comprising RNA residues and terminal blocking groups preventing the blocked ends from ligation by a ligase and/or extension by a polymerase. FIG. 1C: Examples of sequences, secondary structures and compositions of HPAs. Underlined nucleotides are RNA and non-underlined nucleotides are DNA.

FIG. 2A-2D. Schemes to exemplify preparation of ssDNA sequencing libraries using the HASL-free-Seq (Hairpin Adapter Splint-assisted Ligation for range-extended Sequencing) approach. FIG. 2A: Sequential, splint-dependent ligation by a DNA ligase of Hairpin Adapters (3′-HPA and 5′-HPA) to a sample ssDNA having 5′-p and 3′-OH ends, where the 3′-HPA is ligated before ligation of the 5′-HPA. Each hairpin adapter has a random hexanucleotide (N₆) overhang that serves as a ligation splint, and an RNA segment in the stem as shown. The 3′-HPA has a 5′-p, the N₆ overhang at its 3′ end, with a 3′-end blocking group preventing self-ligation between two 3′-HPA molecules. The 5′-HPA has the N₆ overhang with a 5′-OH (or optional blocking group) at its 5′ end, and a 3′-OH as shown. After the first ligation reaction of 3′-HPA to sample ssDNA (Step 1), the remaining unligated 3′-HPA could be blocked to inhibit formation of adapter dimers (e.g., as shown in FIGS. 3A-C). The blocking step is optional. In Step 2, the 5′-HPA is ligated to the ssDNA-3′-HPA ligation products. In Step 3, the newly formed 5′-HPA-ssDNA-3′-HPA ligation products are processed by RNase H (Step 3), which cleaves the RNA containing segments and opens up the hairpin adapters for hybridization with PCR primers. Optionally, the cleaved 5′-HPA-ssDNA-3′-HPA ligation products are cleaned-up to deplete other components and side products of upstream ligation and cleavage reactions before the next step. In Step 4, the cleaved 5′-HPA-ssDNA-3′-HPA ligation products are amplified by PCR using the Illumina sequencing primers followed by a clean-up step to remove unincorporated primers. FIG. 2B: Sequential ligation by a DNA ligase of Hairpin Adapters (5′-HPA and 3′-HPA) to a sample ssDNA that has 5′-p and 3′-OH ends, where the 5′-adapter is ligated before the 3′-HPA. Compositions of the 5′-HPA and 3′-HPA are described in the legend to FIG. 2A. After the first ligation reaction of 5′-HPA to sample ssDNA (Step 1), the remaining unligated 5′-HPA could be blocked to inhibit formation of adapter dimers (e.g., as shown in FIGS. 3A and 3E-F). The blocking step is optional. In Step 2, the 3′-HPA is ligated to the 5′-HPA-ssDNA ligation products. In Step 3, the newly formed 5′-HPA-ssDNA-3′-HPA ligation products are processed by RNase H, which cleaves the RNA containing segments and opens up the hairpin adapters for hybridization with PCR primers. Optionally, the cleaved 5′-HPA-ssDNA-3′-HPA ligation products are cleaned-up to deplete other components and side products of upstream ligation and cleavage reactions before the next step. In Step 4, the cleaved RNase H-treated 5′-HPA-ssDNA-3′-HPA ligation products are amplified by PCR using the Illumina sequencing primers followed by a clean-up step to remove unincorporated primers. FIG. 2C: Sequential ligation by a DNA ligase of Hairpin Adapters (5′-HPA and 3′-HPA) to a sample ssDNA that has 5′-p and 3′-p or 3′-OH ends, where the 5′-HPA is ligated before the 3′-HPA. Compositions of the 5′-HPA and 3′-HPA are described in the legend to FIG. 2A. After the first ligation reaction of 5′-HPA to sample ssDNA (Step 1), the remaining unligated 5′-HPA could be blocked to inhibit formation of adapter dimers (e.g., as shown in FIGS. 3A and 3E-F). The blocking step is optional. In Step 2, the 3′-HPA is ligated to the 5′-HPA-ssDNA ligation products in the presence of a polynucleotide kinase (PNK) that converts 3′-p to 3′-OH at the 3′ end of sample ssDNA before the 3′-HPA ligation. In Step 3, the newly formed 5′-HPA-ssDNA-3′-HPA ligation products are processed by RNase H (Step 3), which cleaves the RNA containing segments and opens up the hairpin adapters for hybridization with PCR primers. Optionally, the processed 5′-HPA-ssDNA-3′-HPA ligation products are cleaned-up to deplete other components and side products of upstream ligation and cleavage reactions before the next step. In Step 4, the cleaved 5′-HPA-ssDNA-3′-HPA ligation products are cleaned-up and then amplified by PCR using the Illumina sequencing primers followed by a clean-up step to remove unincorporated primers. FIG. 2D: Simultaneous ligation by a DNA ligase of Hairpin Adapters (5′-HPA and 3′-HPA) to a sample ssDNA that has 5′-p and 3′-OH ends, where both the 5′-HPA and the 3′-HPA are ligated simultaneously in one step (Step 1). Compositions of the 5′-HPA and 3′-HPA are described in the legend to FIG. 2A. In Step 2, the newly formed 5′-HPA-ssDNA-3′-HPA ligation products are processed by RNase H, which cleaves the RNA containing segments and opens up the hairpin adapters for hybridization with PCR primers. Optionally, the cleaved 5′-HPA-ssDNA-3′-HPA ligation products are cleaned-up to deplete other components and side products of upstream ligation and cleavage reactions before the next step. In Step 3, the cleaved 5′-HPA-ssDNA-3′-HPA ligation products are amplified by PCR using the Illumina sequencing primers followed by a clean-up step to remove unincorporated primer.

FIG. 3A-3F. Schemes to exemplify blocking of unligated first HPA during preparation of ssDNA sequencing libraries using the HASL-free-Seq approach to inhibit adapter dimers formation. FIG. 3A: Formation of adapter dimers by ligation between 5′-HPA and 3′-HPA. FIG. 3B: Optional ligation of remaining unligated 3′-HPA (from Step 1, FIG. 2A) with a blocking oligonucleotide (3′-BON) consisting of a random hexamer (N₆), by a DNA ligase to block 3′-HPA ligation with the 5′-HPA in Step 2 (FIG. 2A). Optionally, unligated 3′-BON is depleted before the 5′-HPA ligation. FIG. 3C: Ligation of remaining unligated 3′-HPA (from Step 1, FIG. 2A) with a hairpin blocking oligonucleotide (3′-BON) having a 3′-overhang, e.g., a random hexamer (N₆), by a DNA ligase to block 3′-HPA ligation with the 5′-HPA in Step 2 (FIG. 2A). Optionally, unligated 3′-BON is depleted before the 5′-HPA ligation. FIG. 3D: Examples of sequences and secondary structures of blocking oligonucleotides (BONs). FIG. 3E: Ligation of remaining unligated 5′-HPA (from Step 1, FIGS. 2B-C) with a blocking oligonucleotide (5′-BON) consisting of a random hexamer (N₆), by a DNA ligase to block 5′-HPA ligation with the 3′-HPA in Step 2 (FIGS. 2B-C). The 5′-BON has a 5′-p as well as a 3′-end blocking group that prevents self-ligation between two 5′-BON molecules. Optionally, the unligated 5′-BON is depleted before the 3′-HPA ligation. FIG. 3F: Ligation of remaining unligated 5′-HPA (from Step 1, FIGS. 2B-C) with a 5′-BON consisting of a hairpin with a 5′-overhang, e.g., a random hexamer (N₆), by a DNA ligase to block 5′-HPA ligation with the 3′-HPA in Step 2 (FIGS. 2B-C). The 5′-BON has a 5′-p as well as a 3′-end blocking group that prevents self-ligation between two 5′-BON molecules. Optionally, unligated 5′-BON is depleted before the 3′-HPA ligation.

FIG. 4A-4C. Comparison of profiles of sequencing libraries made by different variants of HASL-free-Seq for cell-free DNA (cfDNA) isolated from plasma samples. Agilent TapeStation profiles are shown for Illumina sequencing libraries prepared using 0.3 ng cfDNA inputs. The denatured cfDNA having 5′-p and 3′-OH ends were ligated to hairpin adapters (HPAs). Designated fractions of sequencing libraries were labelled as: AD—adapter dimers (products of ligation between two adapters peaked at about 130 nt) containing no cfDNA insert between the adapters; USF—ultrashort fraction containing cfDNA inserts of 20 to 80 nt, and MPF—main peak fraction containing cfDNA inserts peaked at ˜165 nt. FIG. 4A: Library profiles for different orders of adapter ligation to cfDNA isolated from plasma of a healthy donor. In 3′-5′ sequential reaction, the 3′-HPA was ligated in the first ligation step and the 5′-HPA was ligated in the second ligation step as schematically shown in FIG. 2A. In 5′-3′ sequential reaction, the 5′-HPA was ligated in the first ligation step and the 3′-HPA was ligated in the second ligation step as schematically shown in FIGS. 2B-C. In simultaneous reaction scheme, the two adapters were ligated at the same time in one step as schematically shown in FIG. 2D. Unexpectedly, we found that ligation orders could affect yields of the designated fractions. Here, the 5′-3′ sequential ligation provided the highest yield of MPF and USF fractions while the AD fraction was the lowest, whereas the simultaneous ligation provided highest yield of AD. The 3′-5′ sequential ligation yielded the smallest MPF, lower AD fraction and about the same USF fractions in comparison to the simultaneous variant. Relative differences between these profiles could be a result of different duration of ligation between the certain ends of cfDNA and adapters for the different adapter ligation orders, and also by a competition between adapter-adapter and adapter-cfDNA ligation reactions. FIG. 4B: Library profiles for different orders of adapter ligation to cfDNA isolated (in contrast to FIG. 4A) from plasma of a patient diagnosed with breast cancer. Workflow and abbreviations were the same as in FIG. 4A. Here in FIG. 4B, the 3′-5′ sequential ligation provided the highest yields of MPF and USF, whereas both the 3′-5′ sequential and simultaneous ligations provided similar (albeit smaller than 3′-5′) yields of these cfDNA fractions. The simultaneous ligation consistently yielded larger AD fraction than sequential variants. The relative variations between the FIG. 4A and FIG. 4B profiles can be caused by differences in fragmentation level and nucleotide compositions of the cfDNA fragments isolated from the healthy and breast cancer plasma samples. Moreover, the cfDNA having different terminal sequences might ligate with adapters with different efficiency. FIG. 4C: Effect of adapter blocking on library profile for a sequential ligation of adapters to cfDNA isolated from plasma of a patient diagnosed with breast cancer. According to 3′-5′ workflow, 3′HPA was ligated first and 5′-HPA was ligated second (see FIG. 2A). The remaining unligated 3′-HPA was either not blocked (see FIG. 2A) or blocked by a ligation with 3′-BON blocking oligonucleotide (as schematically shown in FIG. 3B) before the 5′-HPA ligation. The latter variant is also described in EXAMPLES 1 and 2. Although the use of 3′-BON resulted in overall reduction of the designated library fraction yields (see legend to FIG. 4A for abbreviations), the drastic reduction in the level of adapter dimers (AD) fraction will significantly increase the proportion of sequencing reads corresponding to the ultrashort fraction (USF) relative to AD, thus providing higher sensitivity and specificity in detecting disease-specific alteration of cfDNA sizes and sequences.

FIG. 5. Comparison of cfDNA fragmentation sequencing profiles for HASL-free-Seq and Competitor A sequencing libraries. The sequencing libraries were prepared using 0.6 ng inputs of cfDNA isolated from plasma of a healthy donor and then subjected to Illumina paired-end sequencing (2×150 nt reads). The HASL-free-Seq libraries were prepared using 3′→5′ workflow without blocking step (see the legend to FIG. 4C and EXAMPLE 3). Competitor A (Accel 1S) libraries were prepared using Accel-NGS® 1S Plus DNA Library Kit and protocol (Swift Biosciences) considered as the benchmark among current, commercially available methods for ssDNA-Seq library preparation. After trimming adapter dimer sequences, sequences were aligned to the human genome (hg19). Then, the aligned sequences were normalized to counts per million (CPM) and were plotted as percent of sequencing reads against cfDNA fragment lengths. The HASL-free-Seq approach detects larger numbers of ultrashort fragments of 20-80 nt and provides higher resolution fragment peaks with ˜10-nt periodicity than Accel 1S. The latter periodicity corresponds to the helical pitch of DNA on the nucleosome core, which protects one strand of the duplex from cleavage by DNase(s) occurring during apoptosis and during circulation of cfDNA in blood, while the main library peak of ˜165 nt is consistent with a footprint of a single nucleosome plus internucleosome spacer. In contrast to HASL-free-Seq, the Accel 1S protocol practically abolishes the sawtooth-like patterns and artificially elongates cfDNA fragments by ˜10 nt at their 3′ end that together could complicate an identification of biologically relevant changes in cfDNA semi-random fragmentation and corresponding sequences of cfDNA fragments.

FIG. 6. Identification of tumor-specific mutations in ultrashort fraction of cfDNA sequencing libraries prepared by HASL-free-Seq and Competitor A methods. The sequencing libraries were prepared using cfDNA isolated from 1 representative plasma sample of a healthy donor and 3 plasma samples isolated from patients diagnosed with different types and stages of breast cancer (post-treatment). These sequencing libraries were subjected to Illumina single read sequencing (150 nt reads). The HASL-free-Seq libraries were prepared using 3′→5′ sequential ligation workflow with blocking step (see the legend to FIG. 4C and EXAMPLE 1). Competitor A (Accel 1S) libraries were prepared as described in EXAMPLE 2. The choice of just one representative healthy sample used here was based on our finding that plasma cfDNA fragmentation sequencing profiles for different healthy donors are quite similar but could significantly vary for different breast cancer patients. By sequencing analysis, we measured numbers of unique mutations in the Estrogen Receptor 1 (ESR1) gene that were identified only in the plasma cfDNA from breast cancer patients, but not in the plasma of a healthy donor. The amounts of these tumor-specific mutations determined by the HASL-free-Seq (H) and Accel 1S (A) methods were plotted as a function of cfDNA length (read length bins). We found that the number of tumor-specific ESR1 mutations detected in short cfDNA fractions decreased in the following fragment lengths order: 51-80-nt>20-50-nt>81-100-nt. HASL-free-Seq can detect significantly more tumor-specific mutations in the ultrashort (20-80 nt) fractions because of its ability to detect ultrashort DNA fragments with higher sensitivity than Accel 1S.

FIG. 7A-7D. Comparison of profiles for cfDNA Methyl-Seq sequencing libraries. FIG. 7A: Comparison of Agilent TapeStation sequencing library profiles for cfDNA samples before (first library) and after (second library) bisulfite treatment (or conversion) using Methyl-Seq variant of HASL-free-Seq protocol (HASL-free-Methyl-Seq) featuring the 5′→3′ sequential ligation (see the legend to FIG. 2C and EXAMPLE 4). Designated fractions of sequencing libraries were labelled as shown in the legend to FIG. 4A. The library prepared with the bisulfite-treated cfDNA showed higher level of DNA fragmentation (increased USF library fraction) and overall lower yields of longer library fractions relative to the non-treated cfDNA profile. FIG. 7B: Comparison of cfDNA fragmentation sequencing profiles for HASL-free-Seq and HASL-free-Methyl-Seq for sequencing libraries described in the legend to FIG. 7A. The fragmentation sequencing profiles plotted as percent of sequencing reads against cfDNA fragment lengths (as described in the legend to FIG. 5 and in EXAMPLE 4) provides quantitative information about increasing numbers of the ultrashort cfDNA fragments as a result of the bisulfite treatment. FIG. 7C: Comparison of Agilent TapeStation profiles for cfDNA sequencing libraries prepared by HASL-free-Methyl-Seq and Competitor B (Pico Methyl-Seq™ Library Prep Kit from Zymo Research) for the same bisulfite-treated cfDNA sample (see EXAMPLE 4). FIG. 7D: Comparison of cfDNA fragmentation sequencing profiles for sequencing libraries described in the legend to FIG. 7C. Dramatic increases of ultrashort and decreases of longer fragments of cfDNA in these sequencing profiles for the Competitor B protocol (shown in FIGS. 7C and 7D) is presumably a result of artificial shortening of the cfDNA fragments and thus inability of this method to incorporate into the sequencing libraries the full lengths of cfDNA fragments present in the bisulfite-treated samples. Evidently, the sequencing library made by HASL-free-Methyl-Seq can capture cfDNA fragments encompassing a wider range of both size and sequence than that of Competitor B for the same bisulfite-treated samples. These features could result in higher sensitivity and specificity in detecting disease-specific cfDNA methylation patterns.

### DETAILED DESCRIPTION

Described herein are compositions and methods for preparation of sample ssDNA sequencing libraries for high-throughput sequencing methods, including (but not limited to) second-generation or Next-Generation Sequencing (NGS) or single-molecule sequencing. Such sequencing libraries can be used for the discovery and detection of one or more DNA molecules in a sample for research and clinical diagnostic purposes.

Sample ssDNAs described herein include naturally occurring and/or synthetic DNA molecules that are either naturally single-stranded DNA (ssDNA) or denatured double-stranded DNA. Among naturally occurring DNA, circulating cell-free DNA (cfDNA) is of particular interest. The cfDNAs, which are found in blood and in most other body fluids (biofluids), represent a promising, minimally invasive (liquid biopsy) source of diagnostic information for cancer, microbial infection and other diseases. cfDNAs are normally present at low concentration in blood but may be elevated in patients with cancer as well as some other disorders such as stroke, microbial infection, myocardial infarction, autoimmune disorders, and pregnancy-associated complications. Tumor-derived fractions of cfDNA (circulating tumor DNA or ctDNA) can be identified by cancer-specific mutations and patterns of methylation as well as generally smaller average fragment size. ctDNA (like mitochondrial and microbial DNA) is more highly degraded than cfDNA from healthy individuals. Thus, short cfDNA fragments of 120 nucleotides or less in length (≤120 nt) are usually significantly elevated in blood samples of cancer patients. Size-selecting short cfDNA fragments prior to sequencing library preparation improves sensitivity of detecting ctDNA. Moreover, bisulfite-conversion chemistry used for detection of cfDNA methylation leads to further degradation of cfDNA fragments. cfDNAs found in urine are also more degraded than in blood. A potentially transformative application of short and ultrashort cfDNA analysis would be detection of microbial infections, cancer at early stages detection and monitoring residual cancer post-treatment.

High-throughput NGS and single-molecule sequencing are generally the preferred technologies for analyzing cfDNA. However, due to the presence of single-strand nicks, the short DNA fragments that comprise cfDNA are not efficiently incorporated into sequencing libraries when they are prepared from non-denatured, double-stranded DNA molecules by conventional DNA-Seq library preparation methods. A few, recently developed methods that use single-stranded (or denatured double-stranded) DNA for the preparation of sequencing libraries (ssDNA-Seq) allow detection of short cfDNA fragments with higher sensitivity than standard DNA-Seq methods.

However, the current commercially available kits for ssDNA-Seq library preparation have certain drawbacks, including (but not limited to): (a) pre-analytical “repairing” of ssDNA ends (e.g., 3′-end dephosphorylation and/or 5′-phosphorylation), which prevents discrimination between DNA fragments having ends with different phosphorylation status (for example, derived from apoptosis vs. other DNA degradation pathways) and may significantly decrease relative concentration of ctDNA in a sample; (b) use of workflows allowing incorporation of RNA fragments (that may contaminate cfDNA preps) into the ssDNA sequencing libraries; (c) employing enzymatic reaction(s) on a solid support, which is less efficient compared to reaction(s) in solution and therefore may reduce the sensitivity of cfDNA detection; (d) use of clean-up protocols to deplete or prevent formation of adapter-dimers lacking a sample ssDNA insert between the adapters that also reduce representation of or render undetectable the shortest (ultrashort) cfDNA fragments (having great biomarker potential) in sequencing libraries; and (e) complicated and laborious workflow and a need for proprietary enzymes (available only from a single source) that makes it more expensive than the standard DNA-Seq library preparation methods.

The COMPOSITIONS and METHODS disclosed herein overcome the drawbacks described above through the following features: (a) a novel hairpin design of sequencing adapters; (b) workflow that preserves naturally occurring DNA phosphorylation status and end sequences; (c) the use of enzymatic (or chemical) steps that disallow incorporation of RNA fragments into the ssDNA sequencing library; (d) highly-efficient, sequential ligation of hairpin adapters to ssDNA fragments in solution, allowing the use of low cfDNA inputs (≤1 ng), even for bisulfite sequencing, since only a few ng of cfDNA can be isolated from typically available volume of biofluid clinical samples (e.g., 1 ml of plasma or serum); (e) optional, ligation-based adapter-blocking strategy that inhibits the formation of adapter dimers; (f) a simple protocol using just non-proprietary enzymes and low material cost.

### Composition(s)

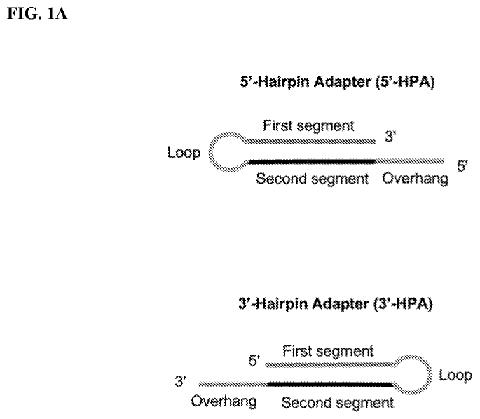

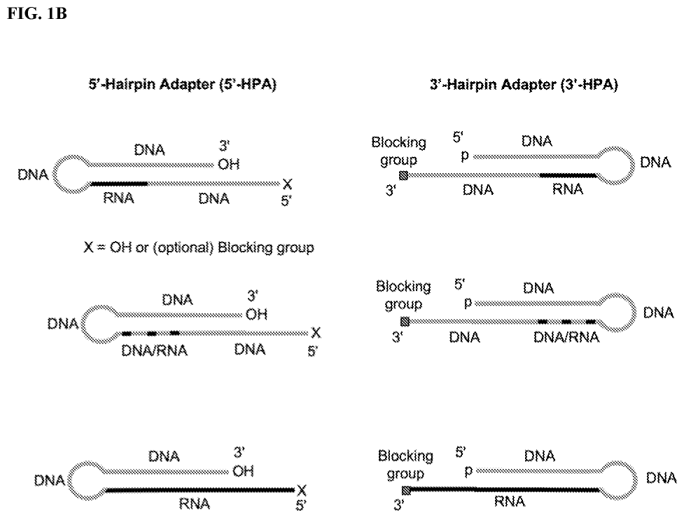

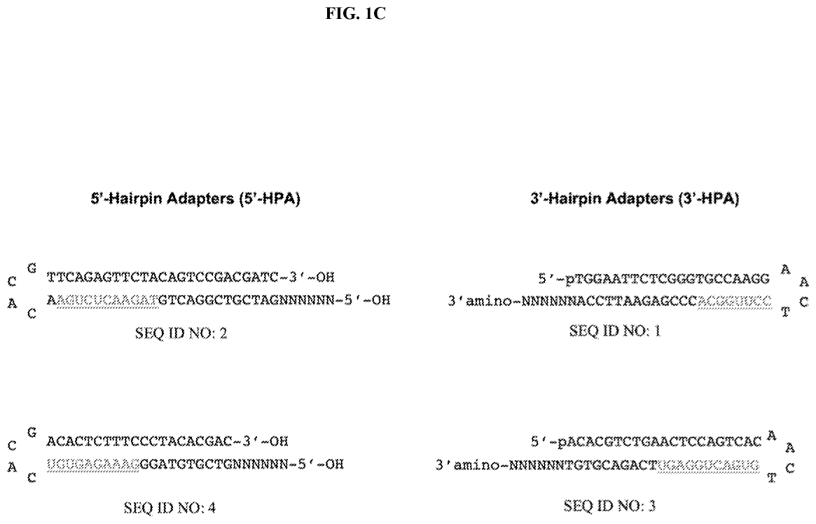

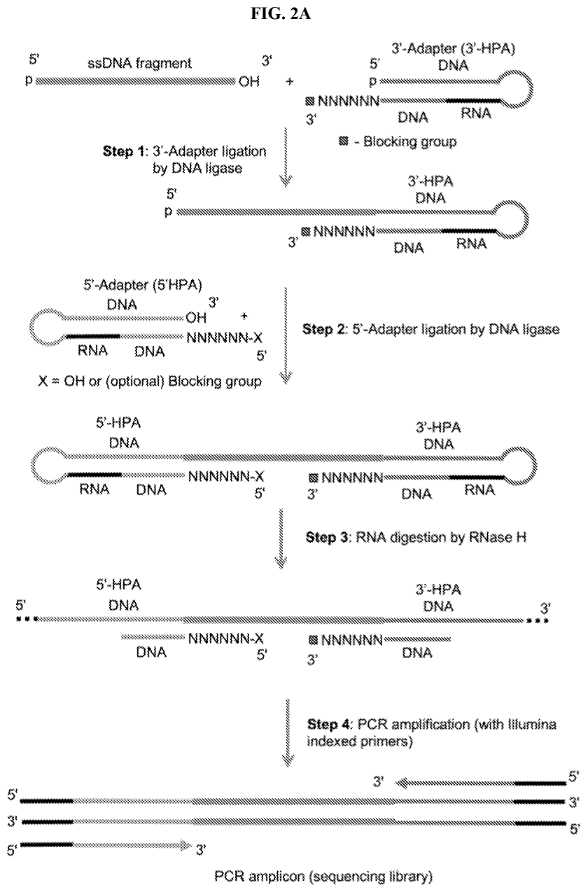

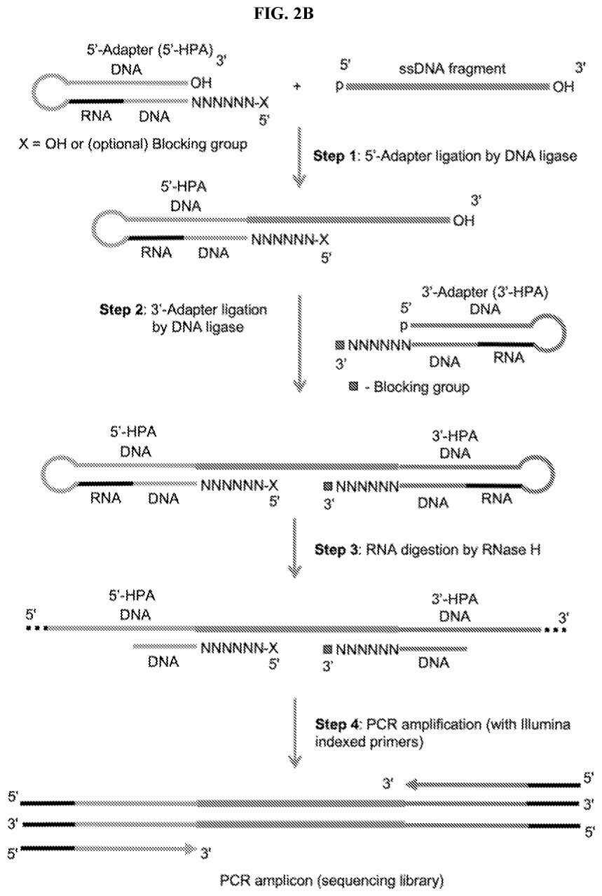

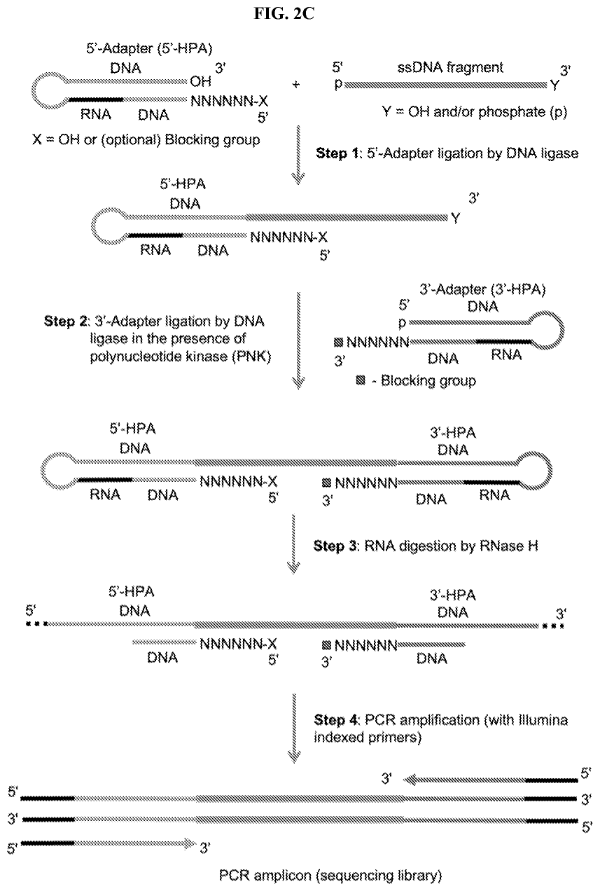

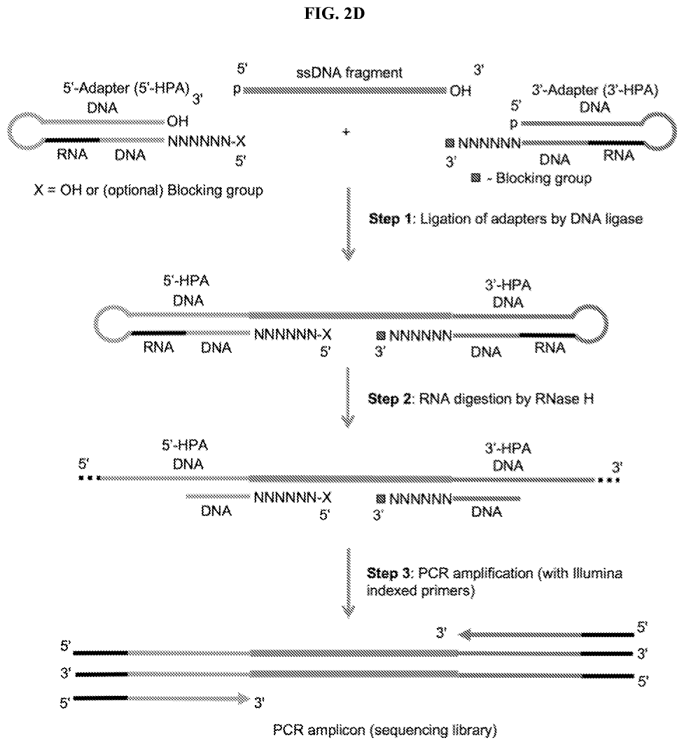

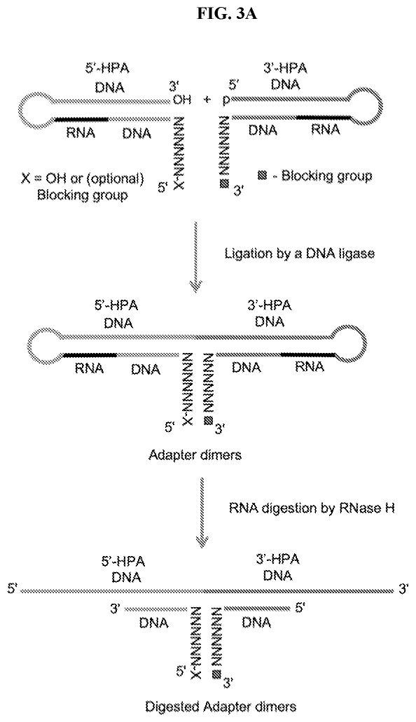

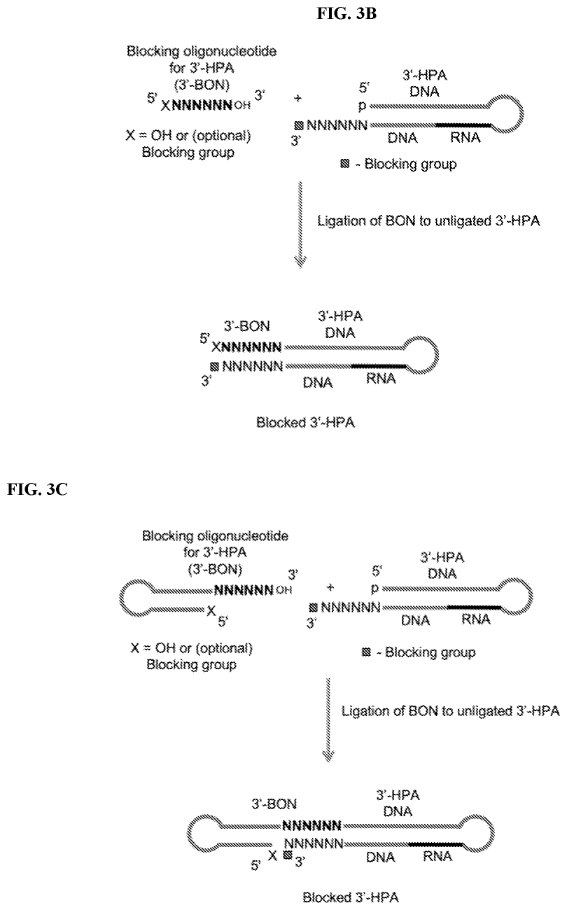

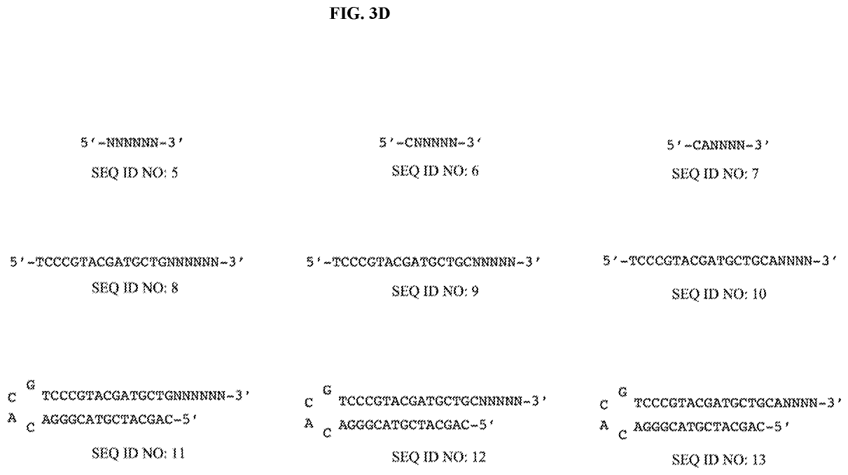

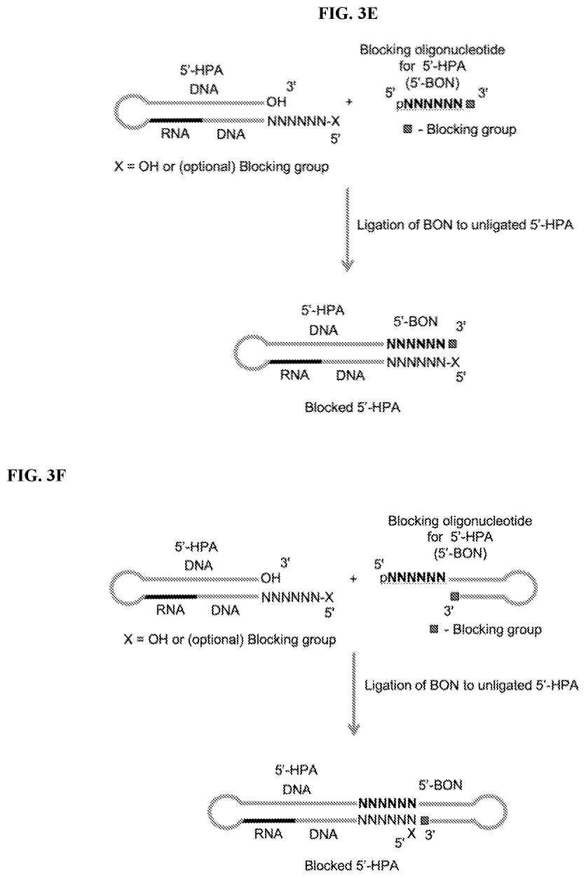

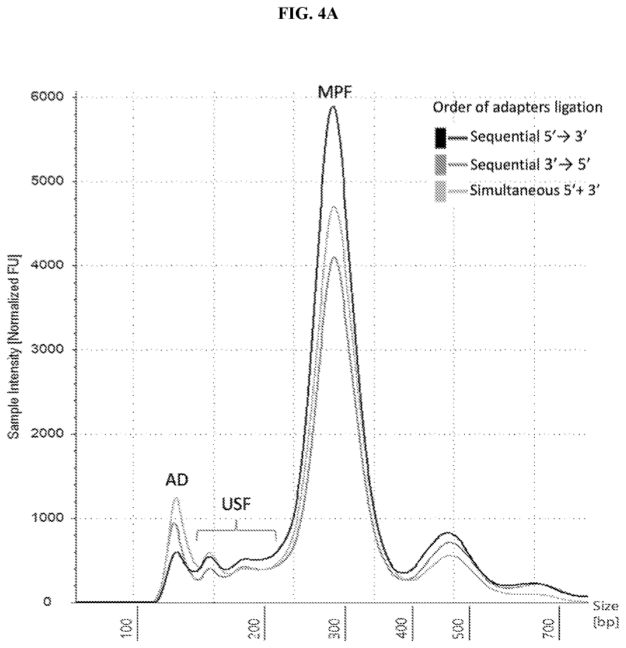

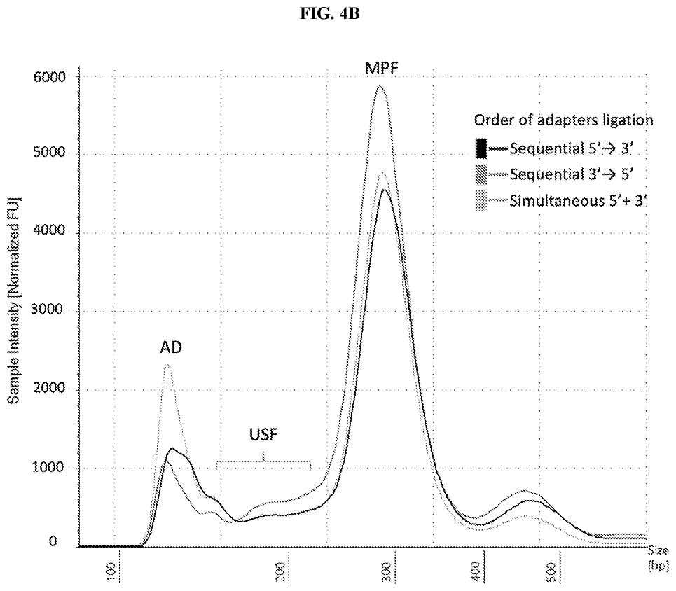

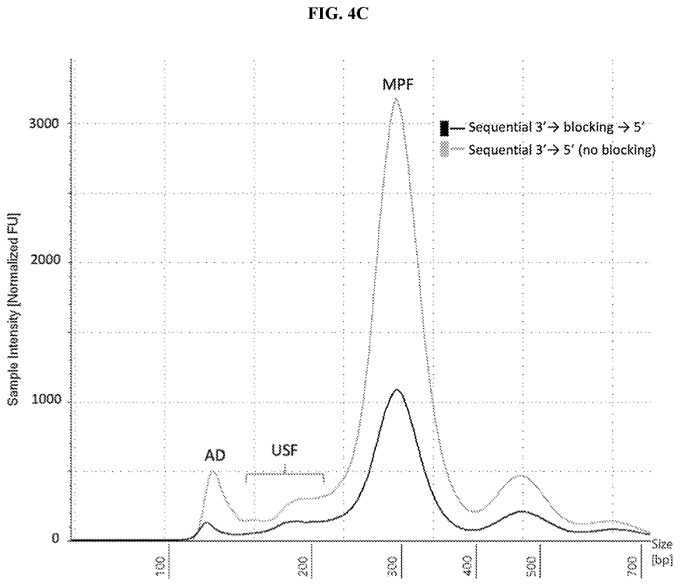

In some embodiments, disclosed herein are compositions suitable for preparation of ssDNA sequencing libraries comprise a plurality of: first hairpin adapter (HPA1), second hairpin adapter (HPA2). In some embodiments, the compositions comprise a plurality of blocking oligonucleotide(s) (BON). Nonlimiting examples of hairpin adapters are depicted in FIGS. 1-2. Nonlimiting examples of BONs are depicted in FIGS. 3B-3F.

In some embodiments, the hairpin adapter (HPA) compositions allow their ligation to the ends of ssDNA. The first HPA (HPA1) may be ligated to the first ssDNA end to produce a HPA1-ssDNA or ssDNA-HPA1 ligation product, and a second HPA (HPA2) may be ligated to the second DNA end of the HPA1-ssDNA or ssDNA-HPA1 ligation product to produce a HPA1-ssDNA-HPA2 or HPA2-ssDNA-HPA1 ligation product. In some embodiments, HPA1 and HPA2 are ligated to ssDNA sequentially (wherein these adapters are ligated in separate ligation steps). In some embodiments, HPA1 and HPA2 are ligated to ssDNA simultaneously (wherein both adapters are ligated to the ssDNA in the same ligation step).

In contrast to double-stranded adapters containing two separate, unconnected adapter and splint strands (or segments), the hairpin adapter structure eliminates the need to anneal the adapter and splint strands, and prevents their dissociation during ligation reactions and heating steps. However, the higher thermostability of secondary (intramolecular) structures of the HPAs relative to the double-stranded adapters can interfere with the intermolecular annealing of the HPAs with PCR and/or sequencing primers. To overcome this problem, in some embodiments, the HPA composition described herein comprise at least one cleavable residue in the segment complementary to the primer specific sequences. This feature provides a partial cleavage (or processing) of HPAs after their ligation to ssDNA. In some embodiments, the cleavage of the HPA moieties of HPA1-ssDNA-HPA2 or HPA2-ssDNA-HPA1 ligation products converts the hairpin adapters to their cleaved forms that are partially single-stranded and, therefore, more accessible (e.g. opened) for hybridization with primer(s) or probes than hairpin or double-stranded adapters. In some embodiments, in opened forms of the cleaved HPAs, the remaining duplex between the adapter and splint segments (see FIGS. 2A-C for a non-limiting example) becomes unstable allowing hybridization with a probe or primer. In some embodiments, conversion of the ligated HPAs may result in more efficient amplification and/or detection of the HPA1-ssDNA-HPA2 or HPA2-ssDNA-HPA1 ligation products.

In some embodiments, the BON may be ligated to HPA1 (but not to HPA2) in the sequential ligation protocol. In some embodiments, after ligation of the HPA1 to the ssDNA, the single BON is ligated to the remaining unligated HPA1 forming an HPA1-BON or BON-HPA1 ligation product to prevent direct ligation between HPA1 and HPA2 (e.g. forming HPA1-HPA2 ligation products (adapter dimers) containing no ssDNA insert). In some embodiments, the compositions of HPA1 and HPA2 disallow ligation between HPA1 and HPA2 ends that are not ligated to ssDNA in the HPA1-ssDNA-HPA2 or HPA2-ssDNA-HPA1 ligation products, to prevent circularization and concatamerization of these products that may interfere with sequencing of these products.

### Hairpin Adapters

The HPAs may comprise nucleic acid residues (DNA, RNA), modified nucleic acid residues, and non-nucleotide residues or a combination thereof. In some embodiments, the HPAs comprise a stem region comprising first and second segments, a loop connecting them, and a single-stranded overhang located at the end of the second segment opposite to the loop. The first and second segments comprise complementary sequences forming a double-stranded stem structure. The overhang can serve as a splint to enable a splint-dependent ligation between the free end (located opposite to the loop) of the first segment and one of the ends of sample ssDNAs to produce IPA-ssDNA ligation product(s).

The end of the HPA may optionally comprise a chemical modification (“blocking group”) that prevents ligation of the HPA by a ligase and/or extension of that end by a polymerase. The first segment (and, optionally, loop) comprises one or more primer-specific and/or probe-specific sequences or sites. A cleavable part of the second segment that is complementary to the first segment comprises one or more cleavable residues located in the stem region of the second segment in vicinity of the loop. In some embodiments, the cleavable part is located at least 1, 2, 3, 4, 5, 6, 7, 8, 9, 10, 11, 12, 13, 14, 15, 16, 17, 18, 19, or 20 nucleotides distance from the junction between the double-stranded stem of the second segment and the loop. In some embodiments, the cleavable part is located at 9 or more nucleotides distant (≥9 nt) from the junction between double-stranded stem and single-stranded overhang. Scission (or cleavage) of the cleavable residues renders the first HPA segments more accessible for hybridization with complementary oligonucleotide primer(s) and/or probes.

In some embodiments, the primer-specific and/or probe-specific sites of the HPAs are at least partially complementary or corresponding to sequences of primer and probes selected from: amplification primers, sequencing primers, detection primers, detection probes, hybridization probes, capture probes, anchor probes, linker probes or combination thereof.

The loop of the HPA may comprise 1 or more residues selected from: nucleotides, modified nucleotides, and non-nucleotide linkers (or moieties), or a combination thereof. In some embodiments, the loop region may comprise from 3 to 9 residues. In some embodiments, the loop region may comprise at least 1, 2, 3, 4, 5, 6, 7, 8, 9, or 10 residues.

The HPA(s) cleavable residues (or moieties) may be selected from: RNA, deoxyuridine (dU), deoxyinosine (dI) nucleotides; internucleotide disulfide (S—S) linker and internucleotide phosphorothioate (PS) bridging or non-bridging linkage.

The HPA stem region may comprise 3 or more nucleotide base pairs. In some embodiments, the HPA stem region comprises at least 3, 4, 5, 6, 7, 8, 9, 10, 11, 12, 13, 14, 15, 16, 17, 18, 19, or 20 nucleotides in length. In some embodiments, the HPA stem may comprise from about 3 to 30,4 to 30,5 to 30,6 to 30,7 to 30,8 to 30,9 to 30, 10 to 30, 11 to 30, 12 to 30,13 to 30, 14 to 30, 15 to 30, 16 to 30, 17 to 30, 18 to 30, 19 to 30, 20 to 30,21 to 30,22 to 30, 23 to 30, 24 to 30 or 25 to 30 base pairs. In some embodiments, the HPA stem may comprise from 18 to 30 base pairs. The HPA stem may comprise DNA, RNA, or a combination thereof. The HPA stem may comprise DNA. The HPA stem may comprise RNA. The HPA stem may compose more DNA and RNA.

The HPA overhangs may comprise one or more randomized (or degenerate) nucleotide residues (N); and defined nucleotide residues (e.g., G, C, A, T) or combination thereof to allow HPA simultaneous ligation with any sample ssDNAs comprising a multitude of different nucleotide sequences at their ends, including unknown and known (or target) sequences. In some embodiments, the HPA overhangs comprise an unknown sequence. In some embodiments, the HPA overhangs comprise a known sequence. In some embodiments, the overhangs comprise at least about 1, 2, 3, 4, 5, 6, 7, 8, 9, 10, 11 or 12 randomizes residues. In some embodiments, the overhangs comprise from 1 to 12 randomized residues selected from: nucleotide residues, modified nucleotide residues, or a combination thereof. In certain embodiments, overhangs comprise 5 or 6 randomized residues.

The HPA overhangs may comprise one or more nucleotide residues selected from: DNA, RNA and modified nucleotide residues or combination thereof. Use of modified nucleotides that can form more stable duplexes than unmodified DNA or RNA nucleotides (e.g., Locked nucleic acids, 2′-OMethyl, 2′-Fluoro, 2-Amino-dA, 5-Methyl-dC, C-5 propynyl-C, C-5 propynyl-U) can be useful for short HPAs with very short overhangs of 1 to 4 nt.

In some embodiments, efficacy of splint-dependent ligation between HPA and ssDNA depends on the composition of the HPA overhangs (including a presence of DNA, RNA or modified nucleotides) that can interfere with binding and ligation activity of certain ligases. For example, T4 DNA ligase and T4 RNA ligase activities are affected by differences in DNA and/or RNA compositions for overhangs and neighboring (to the overhang) nucleotides (e.g., as described in Bullard & Bowater, 2006. Biochem. J. 398: 135-144).

The HPA can be a 3′-HPA that can be ligated to the 3′-end of a sample ssDNA, or a 5′-HPA that can be ligated to the 5′-end of a sample ssDNA. The HPA(s) may comprise 5′-hydroxyl (5′-OH), 5′-phosphate (5′-p), 3′-hydroxyl (3′-OH) or 3′-phosphate (3′-p) ends.

In some embodiments, one of the HPA ends may comprise a 5′-end or 3′-end blocking group(s) that can prevent these ends' ligation and/or extension by a polymerase. The blocking group may be selected from: a 5′-end blocking group (e.g., as 5′-OH, 5′-OMe, 5′-amino, and 5′ biotin linker); or a 3′-end blocking group (e.g., 3′-p, dideoxynucleoside (e.g., 3′-ddC), 3-Inverted dT (idT), 3′-C3 spacer, 3′-amino, and 3′-biotin).

The 3′-HPA may comprise a 5′-p end and a 3′-overhang having a 3′-end blocking group that prevents self-ligation of two 3′-HPA molecules or ligation of the 3′-HPA with the 5′-HPA(s), or extension by a polymerase. The 5′-HPA may comprise a 3′-OH end and a 5′-end overhang comprising a 5′-OH end or a 5′-end blocking group. The first HPA (HPA1) may be the 3′-HPA and the second HPA (HPA2) may be the 5′-HPA. Alternatively, the HPA1 may be the 5′-HPA and the HPA2 may be the 3′-HPA.

### Blocking Oligonucleotide

In some embodiments, after ligation to 3′ end of sample ssDNA, the remaining unligated 3′-HPA may be blocked (or inactivated) by ligating with a 3′-blocking oligonucleotide (3′-BON). The 3′-BON(s) may comprise 3′-OH and a 5′-OH or 5′-end-blocking group. In some other embodiments, after ligation to 5′ end of sample ssDNA, the remaining unligated 5′-HPA may be blocked (or inactivated) by ligating with a 5′-blocking oligonucleotide (5′-BON). The 5′-BON(s) may comprise 5′-p and a 3′-end-blocking group.

The BON(s), including 3′-BON and 5′-BON, may comprise nucleotide compositions selected from: DNA and/or RNA; randomized (or degenerate) nucleotide residues (N); or defined nucleic acid and/or modified nucleic acid residues; or combination thereof that are at least partially complementary to the HPAs. The BON(s) may comprise from 1 to 12 randomized nucleotides.

The BON(s) structure may be single-stranded (unfolded); or double-stranded oligonucleotide(s); or hairpin structure, or combination thereof (see FIG. 3D for certain non-limiting examples). In some embodiments, the BON is single-stranded. In some embodiments, the BON is double-stranded. In some embodiments, the BON comprises a hairpin structure.

In some embodiments, the BON length may be from about 1 to 40 nucleotides, 5 to 40 nucleotides, 10 to 40 nucleotides, or 30 to 40 nucleotides. In some other embodiments, the BON(s) of 21 nt may comprise a defined (fixed) sequence of 15 nucleotides followed by a sequence of 6 random nucleotides. In yet other embodiments, the BON(s) may comprise a defined (fixed) sequence of 12 to 15 nucleotides followed by sequences containing from 3 to 6 random nucleotides. In some other embodiments, the end of the BON(s) may comprise sequences selected from 4 to 6 randomized nucleotides. The BONs may contain only 4 to 6 randomized nucleotides. In some embodiments, BONs are randomized hexamer (N₆) comprising DNA or RNA.

The BON(s) may be a hairpin oligonucleotide comprising a stem region that comprises first and second segments, a loop connecting one end of first and one end of second segments, and single-stranded overhang located at the end of the second segment opposite to the loop. The first and second segments comprise complementary segments forming a double-stranded stem structure. The overhang can serve as a splint to provide a splint-dependent ligation between the first BON segment(s) end and the 5′-p end(s) of 3′-HPA(s) or to 3′-OH end(s) of 5′-HPA(s) to produce BON-3′-HPA or 5′-HPA-BON ligation product(s), respectively.

The 3′-BON(s) may comprise 3′-OH and a 5′-end-blocking group(s). The 5′-BON(s) may comprise 5′-p and a 3′-end-blocking group(s). The end blocking group prevents this end's ligation and/or extension by a polymerase. The 3′-end blocking groups may be selected from: 3′-p, dideoxynucleoside (e.g., 3′ddC), 3′ Inverted dT (idT), 3′ C3 spacer, 3′ amino, and 3′ biotin linker. The 5′-end blocking group may be selected from: 5′-OH, 5′-amino, 5′-OMethyl and 5′ biotin linker.

Sample ssDNA

The sample ssDNA described herein may comprise synthetic DNA and/or naturally occurring molecules selected from: single-stranded DNA (ssDNA), fragmented ssDNA (or ssDNA fragments), denatured double-stranded DNAs and DNA fragments (or denatured double-stranded DNA fragments). The synthetic DNA may include primers, adapters, linkers, ladders, gene fragments, aptamers, antisense agents, DNA origami, and molecules used for DNA computing and information encoding. The naturally occurring DNA may include circulating cell-free DNA (cfDNA) from a biofluid such as circulating normal genomic DNA, circulating tumor DNA, circulating microbial DNA, circulating bacterial DNA, circulating viral DNA, and circulating mitochondrial DNA; fragmented (or degraded) DNA molecules present in biofluids (e.g., whole blood, plasma, serum, saliva and urine), FFPE samples, and ancient organisms; highly fragmented DNA from ancient organisms or from forensic biological samples.

The sample ssDNA may comprise isolated total nucleic acids including both DNA and RNA, or may comprise only isolated (and/or purified) total DNA. The sample ssDNA may be isolated (or purified) DNA of interest (target DNA).

The target DNAs may be isolated (captured or enriched) by a hybridization with either target-specific oligonucleotide probes in solution followed by attachment of target-probe complexes to a solid support; or on a solid support comprising immobilized target-specific oligonucleotide probes. In some embodiments, the target-specific oligonucleotide probes may comprise or contain RNA and/or other cleavable residues described herein. The target DNA may require a denaturation before the hybridization with the probes. The target DNAs may be captured directly from biofluid(s) (e.g., from whole blood, plasma, serum, saliva, or urine) or from biofluids that have been treated by lysis solutions (“lysed” biofluids) to dissociate DNA complexes with proteins and/or lipids. In some embodiments, the target DNAs are isolated in solution.

The sample ssDNA may have ends selected from: 5′-p and 3′-OH, 5′-OH and 3′-p, or combination thereof (e.g., 5′-p/3′-OH; 5′-OH/3′-p; 5′-OH/3′-OH and 5′-p/3′-p). Ligation of the HPAs to sample ssDNA comprising a fraction of 5′-OH and 3′-p ends may be performed without or with their conversion (“repair”) of these ends into ligatable 5′-p and 3′-OH ends. In some embodiments, such conversion may be performed by a polynucleotide kinase.

The sample ssDNAs may be chemically and/or enzymatically treated (converted or repaired). Chemical and/or enzymatic treatment may occur before or simultaneously with ligating HPA1 and/or HPA2. The sample ssDNAs may be subjected to a bisulfite treatment that can convert unmethylated cytosine (C) to deoxyuridine (dU) residues before ligation. The sample ssDNA may be ligated to HPA1 and/or HPA2 without the pre-treatment (or “repair”) to preserve original (or naturally occurring) ssDNA ends.

The sample ssDNAs may comprise short DNA fragments of 120 or fewer nucleotides in length. The sample ssDNA may contain ultrashort DNA fragments in the range of 20 to 80 nucleotides in length. The sample ssDNA may contain ultrashort DNA fragments in the range of 18 to 50 nucleotides in length. The sample ssDNA may contain long DNA fragments of more than 120 nucleotides in length. The sample ssDNA may contain short and long DNA fragments. In some embodiments, the sample ssDNAs may comprise DNA fragments of no more than about 300, 250, 200, 190, 180, 170, 160, 150, 140, 130, 120, 110, 100, 90, 80, 70, 60, 50, 40, 30,20 or 10 nucleotides in length. In some embodiments, the sample ssDNAs may comprises DNA fragments of at least about 300, 250, 200, 190, 180, 170, 160, 150, 140, 130, 120, 110, 100, 90, 80, 70, 60, 50, 40, 30, 20 or 10 nucleotides in length.

### Methods

In the methods described herein, HPA1 and HPA2 may be ligated to ssDNA either sequentially (wherein these adapters are ligated in separate ligation steps) or simultaneously (wherein both adapters ligated to ssDNA at the same ligation step). In some embodiments, HPA1 and HPA2 are ligated to ssDNA sequentially. In some embodiments, HPA1 and HPA2 are ligated to ssDNA simultaneously. The simultaneous ligation workflow (or protocol) is quick and simple, but it may produce larger amounts of adapter dimer (HPA1-HPA2) sequences that have no ssDNA inserts than the sequential ligation protocols. The adapter dimers may saturate sequencing library and, therefore, reduce numbers of ssDNA sequencing reads. The adapter dimers are usually removed or depleted during preparation of sequencing libraries. However, depleting adapter dimers by commonly used clean-up protocols is usually incomplete and may result in complete or substantial loss of sequencing library fractions comprising ultrashort ssDNA inserts. Moreover, the simultaneous ligation workflow does not allow a use of the adapter blocking that may prevent (or significantly reduce) formation of adapter dimers. The simultaneous workflow may be used for preparation of sequencing libraries from large inputs of sample ssDNA (e.g., ≥1 ng), where the percentage of adapter dimers will be smaller than for libraries prepared with low amounts of sample ssDNA.

In some embodiments, the sequential ligation of ssDNA with HPA1 and HPA2 allows the preparation of sequencing libraries containing higher relative yields of ultrashort ssDNA inserts and lesser yields of adapter dimers than the simultaneous protocol (even without the blocking of remaining unligated HPA1). In some embodiments, the sequential ligation provides higher sequencing library yields than simultaneous ligation workflow. Moreover, the sequential ligation workflows described herein provide higher sequencing library yields for ultrashort cfDNAs and for bisulfite-treated cfDNA in comparison to benchmark methods.

In some embodiments, the methods include ligating the HPA1 described herein to the first end of a plurality of sample ssDNAs to produce a mixture comprising a plurality of HPA1-ssDNA (5′-HPA1-ssDNA-3′) or ssDNA-HPA1 (5′-ssDNA-HPA1-3′) ligation products and remaining unligated HPA1. In some embodiments, the methods include ligating the HPA2 described herein to the second end of the HPA1-ssDNA or ssDNA-HPA1 ligation products to produce HPA1-ssDNA-HPA2 or HPA2-ssDNA-HPA1 (5′-HPA1-ssDNA-HPA2-3′ or 5′-HPA2-ssDNA-HPA1-3′) ligation products, wherein the sample ssDNA is positioned between (or flanked by) the HPA1 and HPA2. In some embodiments, the methods include cleaving the HPA1 and HPA2 to produce cleaved HPA1-ssDNA-HPA2 or HPA2-ssDNA-HPA1 ligation products, wherein the cleaving makes the first segments of HPA1 and HPA2 more accessible for hybridization with primer(s) and/or probe(s). In some embodiments, the methods include hybridizing a first primer comprising a sequence at least partially complementary to the first HPA(s) segment ligated to 3′-end of ssDNA in the cleaved 5′-HPA1-ssDNA-HPA2-3′ or 5′-HPA2-ssDNA-HPA1-3′ ligation products; and extending the primer with a polymerase to produce a plurality of the first primer extension products. In some embodiments, the methods include hybridizing a second primer comprising a sequence at least partially complementary to the 3′-end of the first primer extension products comprising a sequence complementary to the first HPA segment ligated to 5′-end of ssDNA; and extending the primer with a polymerase to produce a plurality of the second primer extension products. In some embodiments, the first primer extension products comprise a sequencing library. In some embodiments, the second primer extension products comprise a sequencing library. In some embodiments, the first and the second primer extension products comprise a sequencing library.

Such methods may include the following general steps. Step 1: Ligating the HPA1 described herein to the first end of a plurality of sample ssDNAs to produce a mixture comprising a plurality of HPA1-ssDNA (5′-HPA1-ssDNA-3′) or ssDNA-HPA1 (5′-ssDNA-HPA1-3′) ligation products and remaining unligated HPA1. Step 2: Ligating the HPA2 described herein to the second end of the HPA1-ssDNA or ssDNA-HPA1 ligation products to produce HPA1-ssDNA-HPA2 or HPA2-ssDNA-HPA1 (5′-HPA1-ssDNA-HPA2-3′ or 5′-HPA2-ssDNA-HPA1-3′) ligation products, wherein the sample ssDNA is positioned between (or flanked by) the HPA1 and HPA2. Step 3: Cleaving the HPA1 and HPA2 to produce cleaved HPA1-ssDNA-HPA2 or HPA2-ssDNA-HPA1 ligation products, wherein the cleaving makes the first segments of HPA1 and HPA2 more accessible for hybridization with primer(s) and/or probe(s). Step 4: Hybridizing a first primer comprising a sequence at least partially complementary to the first HPA(s) segment ligated to 3′-end of ssDNA in the cleaved 5′-HPA1-ssDNA-HPA2-3′ or 5′-HPA2-ssDNA-HPA1-3′ ligation products; and extending the primer with a polymerase to produce a plurality of the first primer extension products. Step 5: Hybridizing a second primer comprising a sequence at least partially complementary to the 3′-end of the first primer extension products comprising a sequence complementary to the first HPA segment ligated to 5′-end of ssDNA; and extending the primer with a polymerase to produce a plurality of the second primer extension products comprising a sequencing library.

Such methods also may include the following options. The HPA1 may be ligated first and HPA2 may be ligated second in two sequential ligation reactions with sample ssDNA. The HPA2 may be used in molar excess over the HPA1. The methods may include simultaneous ligation of HPA1 and HPA2 to the first and the second ends of sample ssDNA. The first HPA (HPA1) may be the 3′-HPA and the second HPA (HPA2) may be the 5′-HPA. The first HPA (HPA1) may be the 5′-HPA and the second HPA (HPA2) may be the 3′-HPA. In some embodiments, the HPA1 remaining unligated to ssDNA may be ligated with the BON to produce HPA1-BON or BON-HPA1 (5′-HPA1-BON-3′ or 5′-BON-HPA1-3′) ligation product(s) before the ligation of a HPA2, thereby disallowing ligation of HPA1 to HPA2 and preventing formation of adapter dimers (products of HPA1 and HPA2 ligation). In certain embodiments, the BON(s) may be used in molar excess over of the first HPA during the ligation. In some embodiments, after ligation to 3′ end of sample ssDNA, the remaining unligated 3′-HPA may be blocked (or inactivated) by ligating with a 3′-BON. In some other embodiments, after ligation to 5′ end of sample ssDNA, the remaining unligated 5′-HPA may be blocked (or inactivated) by ligating with a 5′-BON.

The method may further include amplification of the sequencing libraries described herein to produce an amplified sequencing library. The method may further include a combination of primer extension with the amplification performed simultaneously in one step by PCR. The amplification of the sequencing library may be performed by PCR using a thermostable DNA polymerase. In some embodiments, thermostable DNA polymerase is selected from: LongAmp HotStart Taq DNA polymerase, KAPA HiFi HotStart DNA polymerase, KAPA HiFi HotStart Uracil+DNA polymerase; Pfu Turbo Cx HotStart DNA polymerase. In another embodiments, the amplification may be performed using an isothermal amplification method.

The method may further include a clean-up of the cleaved HPA1-ssDNA-HPA2 or HPA2-ssDNA-HPA1 ligation products to deplete or remove other components and side products of upstream ligation and cleavage reactions (including adapter dimers and polymerase inhibitors) before the primer extension and amplification steps. In some embodiments, the method further comprises performing a post-amplification clean-up to purify the amplified sequencing library from non-extended primers and other components of extension and/or amplification reactions. The clean-up steps could be performed using SPRIselect bead kit (Beckman Coulter) or NGS Library Clean-up kit (YouSeq) or Sephadex G-25 microspin columns.

These methods may use one or more (a plurality or pool of) sequence variants of the first HPA (HPA1). These methods may use one or more (a plurality or pool of) versions of the second HPA (HPA2). These methods may use one or more (a plurality or pool of) sample ssDNA molecules. These methods may employ various techniques for ligating ssDNA to HPA1 and to HPA2, blocking unligated HPA1, cleaving ligated HPA1 and HPA2, and for amplifying HPA1-ssDNA-HPA2 or HPA2-ssDNA-HPA1 ligation products.

As demonstrated in EXAMPLES 2 to 4, the methods described herein have the ability to efficiently incorporate or capture short cfDNA fragments of 120 or fewer nucleotides in length along with longer fragments into sequencing libraries. In some embodiments, the methods can capture ultrashort cfDNA fragments in the length range of 20-80 nt more efficiently than currently available ssDNA-Seq and conventional DNA-Seq methods for preparing sequencing libraries. In some embodiments, the methods can capture ultrashort cfDNA fragments in the length range of 18-50 nt more efficiently than currently available ssDNA-Seq and conventional DNA-Seq methods for preparing sequencing libraries.

The ligating of sample ssDNA with HPA(s) and the ligating of HPA(s) with BON(s) may be performed via splint-dependent (or template-dependent) ligation by a ligase. The ligase may be selected from: T4 DNA ligase, T3 DNA ligase, T7 DNA ligase, PBCV1 DNA ligase, E. coli DNA ligase, human DNA ligase III, T4 RNA ligase 2 and SplintR® Ligase.

The ligating of sample ssDNA with HPA(s) and the ligating of HPA(s) with BON(s) may be performed via splint-independent ligation by a ligase.

The ligation with a DNA ligase may be performed under reaction conditions that favor splint-dependent ligation of sticky DNA ends rather than blunt-end ligation. That may reduce formation of adapter dimers forming three-way junctions. An example of an adapter dimer forming three-way junctions is depicted in FIG. 3A. For this purpose, the ligating by T4 DNA ligase may be performed in the presence of ATP at a concentration selected from the range 50-100 μM. In some embodiments, the ATP may be in a concentration of at least about 30 μM, 40 μM, 50 μM, 60 μM, 70 μM, 80 μM, 90 μM or 100 μM.

In some embodiments, resolvase endonucleases (resolvases) may be used to reduce or inhibit formation of such adapter dimers. In some embodiments, resolvase endonucleases, which cleave branched DNAs including three-way junctions. In some embodiments, resolvases could be present during the ligation reactions performed by a DNA ligase to prevent the DNA ligase from binding to three-way junction complexes formed by unligated adapters. In other embodiments, the resolvases could be used after the ligation of adapters to cleave any branched adapter dimers. The resolvase may be selected from: T7 Endonuclease I, T4 Endonuclease VII and Chaetomium thermophilum GEN1.

Alternatively, the BON depletion may be performed by selective degradation of the BON. In some embodiments, the selective degradation of a single-stranded segment of remaining, unligated 3′-BON may be performed using a 3′-end exonuclease such as Exonuclease T, that preferentially cleaves single-stranded DNA and RNA. In some other embodiments, wherein a BON's single-stranded overhang(s) comprises RNA nucleotides, selective degradation (or cleavage) of the overhang(s) may be performed using a single-strand-specific ribonuclease or other RNA cleaving agents.

In some embodiments, reducing (or inhibiting) formation of adapter dimers may be performed by selective degradation of the HPA1 using exonucleases that will not degrade HPA1-ssDNA or ssDNA-HPA1 ligation products. Selective degradation of the single-stranded overhang of remaining unligated 3′-HPA may be performed using a 3′-end exonuclease such as Exonuclease T that preferentially cleaves single-stranded DNA and RNA. The selective degradation of the single-stranded overhang of remaining, unligated 5′-HPA may be performed using a DNA polymerase having 5′-end exonuclease activity, including without limitations DNA Polymerase I, Large (Klenow) Fragment (KF). In some other embodiments, the selective degradation (or cleavage) of a single-stranded overhang(s) of a HPA1 comprising RNA nucleotides may be performed using a single-strand-specific ribonuclease.

The HPA(s) comprising one or more RNA residues (or moieties) may be cleaved by a cleaving agent. The cleaving agents may be selected from: a ribonuclease, a ribozyme, a deoxyribozyme, basic buffer solutions, alkaline solutions, divalent or multivalent metallic cations, or combinations thereof. In some embodiments, the cleavage of RNA residues may be performed at elevated temperature under conditions preventing DNA damage and/or degradation. The ribonuclease may be selected from members of the RNase H family. The single-strand-specific ribonuclease may be selected from: Ribonuclease I or Ribonuclease I_{f}.

In some embodiments, the use of the RNA cleaving agent for the HPA cleavage may simultaneously cleave any RNA present in the sample ssDNA to prevent incorporation of RNA sequences into the ssDNA sequencing library, thereby producing an RNA-free DNA sequencing library. The RNA cleaving agent may use oligonucleotides comprising sequence-specific or randomized nucleotides to guide a cleavage of RNA molecules incorporated into the ssDNA sequencing library. The RNA cleavage step prevents incorporation of any RNA that could be present in total DNA preps, total nucleic acid isolates, or spiked into sample ssDNA as a carrier RNA, into the sequencing libraries. Eliminating the need to remove RNA from total sample DNA preparations (DNA prep) may simplify the isolation procedure workflow and reduce the loss of short and low-abundant sample DNA fragments.

The use of spike-in RNAs or addition of carrier RNA to samples with very low DNA content (e.g., single-cell lysates or samples of low-abundance target ssDNA isolated by hybridization-based capture methods) could prevent irreversible depletion (or loss) of DNA molecules from such samples by absorption to surfaces (e.g., pipette tips, tubes, wells, slides, beads, or columns) during the preparation of sequencing libraries. In some embodiments, this may provide the ability to efficiently incorporate (or capture) lower inputs of sample ssDNA into sequencing libraries in comparison to currently available methods.

In another embodiment, the method for preparing a sequencing library may comprise ligating the 3′-HPA described herein to the 3′-ends of the plurality of sample ssDNAs to produce a mixture comprising a plurality of ssDNA-3′-HPA ligation products. In some embodiments, the 3′-HPA comprises 2 or more RNA residues located at least 9 nucleotides distance from the free end of its second segment in the stem region. In some embodiments, the 3′-HPA comprises 5 or 6 randomized nucleotide residues at its 3′-end and a 3′-end blocking group. In some embodiments, the methods comprises ligating the 5′-HPA described herein to the 5′ ends of the ssDNA. In some embodiments, the method comprises ligating the 5′ HPA described herein to the 5′ end of the ssDNA-3′-HPA ligation products to produce 5′-HPA-ssDNA-3′-HPA ligation products. In some embodiments, the 5′-HPA comprises 2 or more RNA residues located at least 9 nucleotides distance from the free end of its second segment in the stem region. In some embodiments, the 5′-HPA overhang comprises 5 or 6 randomized nucleotide residues at its 5′-end or a 5′-end blocking group. In some embodiments, the 3′-HPA is ligated to the 3′ ends of the plurality of sample ssDNAs and the 5′HPA is ligated to the 5′ end of the plurality of the sample ssDNAs simultaneously. In some embodiments, the 3′-HPA is ligated to the 3′ end of the plurality of sample ssDNAs before the 5′ HPA is ligated to the 5′ end of the plurality of sample ssDNAs. In some embodiments, the methods comprise cleaving the RNA residues in the 5′-HPAs and 3′-HPAs by treatment with RNase H to produce cleaved 5′-HPA-ssDNA-3′-HPA ligation products. In some embodiments, the cleaving makes the first segments of 5′-HPA and 3′-HPA more accessible for hybridization with primers. In some embodiments, the methods comprise performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification. In some embodiments, the methods comprise amplifying the cleaved 5′-HPA-ssDNA-3′-HPA ligation products by a PCR using a pair of the first and the second sequencing primers to produce (amplified) sequencing library. In some embodiments, the first sequencing primer is at least partially complementary to the first 3′-HPA segment located at the 3′ end of the cleaved 5′-HPA-ssDNA-3′-HPA ligation products. In some embodiments, the second sequencing primer is at least partially corresponding to the first 5′-HPA segments located at the 5 ‘end of the cleaved 5’-HPA-ssDNA-3′-HPA ligation products. In some embodiments, the second primer comprising a sequence at least partially complementary to the 3′-end of the first primer extension products comprising a sequence complementary to the first HPA segment of 5′-HPA. In some embodiments, the methods comprise performing a post-amplification clean-up to purify the amplified sequencing library from non-extended primers and other components of extension and/or amplification reactions to produce purified sequencing libraries that are ready for sequencing.

In another embodiment, the method for preparing a sequencing library may comprise ligating the 5′-HPA described herein to the 5′-ends of the plurality of sample ssDNAs to produce a mixture comprising a plurality of 5′-HPA-ssDNA ligation products. In some embodiments, the 5′-HPA comprises 2 or more RNA residues located at least 9 nucleotides distance from the free end of its second segment in the stem region. In some embodiments, the 5′-HPA comprises 5 or 6 randomized nucleotide residues at its 5′-end. In some embodiments, the methods comprise ligating the 3′-HPA described herein to the 3′ ends of the ssDNA. In some embodiments, the 3′-HPA is ligated to the 3′ end of the 5′-HPA-ssDNA ligation products to produce 5′-HPA-ssDNA-3′-HPA ligation products. In some embodiments, the 3′-HPA comprises 2 or more RNA residues located at least 9 nucleotides distance from the free end of its second segment in the stem region. In some embodiments, the 3′-HPA overhang comprises 5 or 6 randomized nucleotide residues at its 3′-end and a 3′-end blocking group. In some embodiments, the 3′-HPA is ligated to the 3′ ends of the plurality of sample ssDNAs and the 5′HPA is ligated to the 5′ end of the plurality of the sample ssDNAs simultaneously. In some embodiments, the 3′-HPA is ligated to the 3′ end of the plurality of sample ssDNAs before the 5′ HPA is ligated to the 5′ end of the plurality of sample ssDNAs. In some embodiments, the methods comprise cleaving the RNA residues in the 5′-HPAs and 3′-HPAs by treatment with RNase H to produce cleaved 5′-HPA-ssDNA-3′-HPA ligation products. In some embodiments, the cleaving makes the first segments of 5′-HPA and 3′-HPA more accessible for hybridization with primers. In some embodiments, the methods comprise performing a pre-PCR clean-up of the cleaved 5′-HPA-ssDNA-3′-HPA ligation products to deplete or remove other components and side products of upstream ligation and cleavage reactions (including without limitations adapter dimers 5′-HPA-3′-HPA and inhibitors of the PCR) before the PCR amplification. In some embodiments, after ligation to 3′ end of sample ssDNA, the remaining unligated 3′-HPA may be blocked (or inactivated) by ligating with a 3′-BON. In some other embodiments, after ligation to 5′ end of sample ssDNA, the remaining unligated 5′-HPA may be blocked (or inactivated) by ligating with a 5′-BON. In some embodiments, the methods comprise amplifying the cleaved 5′-HPA-ssDNA-3′-HPA ligation products by a PCR using a pair of the first and the second sequencing primers to produce (amplified) sequencing library. In some embodiments, the first sequencing primer is at least partially complementary to the 3′-HPA segment of the cleaved 5′-HPA-ssDNA-3′-HPA ligation products. In some embodiments, the second sequencing primer is at least partially corresponding to the 5′-HPA segments of the cleaved 5′-HPA-ssDNA-3′-HPA ligation products. In some embodiments, the second primer comprising a sequence at least partially complementary to the 3′-end of the first primer extension products. In some embodiments, the performing a post-amplification clean-up to purify the amplified sequencing library from non-extended primers and other components of extension and/or amplification reactions to produce purified sequencing libraries that are ready for sequencing.

In yet another embodiments, ligating the 5′-HPA described herein to the 5′-ends of the plurality of sample ssDNAs to produce a mixture comprising a plurality of 5′-HPA-ssDNA ligation products. In some embodiments, the 5′-HPA comprises 2 or more RNA residues located at least 9 nucleotides distance from the free end of its second segment in the stem region. In some embodiments, the 5′-HPA comprises 5 or 6 randomized nucleotide residues at its 5′-end. In some embodiments, the methods comprise ligating the 3′-HPA described herein to the 3′ ends of the 5′-HPA-ssDNA ligation products in the presence of polynucleotide kinase (PNK) to produce 5′-HPA-ssDNA-3′-HPA ligation products. In some embodiments, the 3′-HPA comprises 2 or more RNA residues located at least 9 nucleotides distance from the free end of its second segment in the stem region. In some embodiments, the 3′-HPA overhang comprises 5 or 6 randomized nucleotide residues at its 3′-end and a 3′-end blocking group. In some embodiments, the methods comprise cleaving the RNA residues in the 5′-HPAs and 3′-HPAs by treatment with RNase H to produce cleaved 5′-HPA-ssDNA-3′-HPA ligation products. In some embodiments, the cleaving makes the first segments of 5′-HPA and 3′-HPA more accessible for hybridization with primers. In some embodiments, the methods comprise performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification. In some embodiments, after ligation to 3′ end of sample ssDNA, the remaining unligated 3′-HPA may be blocked (or inactivated) by ligating with a 3′-BON. In some other embodiments, after ligation to 5′ end of sample ssDNA, the remaining unligated 5′-HPA may be blocked (or inactivated) by ligating with a 5′-BON. In some embodiments, the methods comprise amplifying the cleaved 5′-IPA-ssDNA-3′-HPA ligation products by a PCR using a pair of the first and the second sequencing primers to produce (amplified) sequencing library. In some embodiments, the first sequencing primer is at least partially complementary to the 3′-HPA segment of the cleaved 5′-HPA-ssDNA-3′-HPA ligation products. In some embodiments, the second sequencing primer is at least partially corresponding to the 5′-HPA segments of the cleaved 5′-HPA-ssDNA-3′-HPA ligation products. In some embodiments, the methods comprise performing a post-amplification clean-up to purify the amplified sequencing library from non-extended primers and other components of extension and/or amplification reactions to produce purified sequencing libraries that are ready for sequencing.

In yet another embodiment, the method for preparing a sequencing library may comprise: ligating a 5′-hairpin adapter (5′-HPA) described herein to the 5′-ends of the plurality of sample single-stranded DNA (sample ssDNAs) to produce a mixture comprising a plurality of 5′-HPA-ssDNA ligation products. In some embodiments, the 5′-HPA is comprised of a plurality of residues selected from: nucleic acid residues (DNA and RNA), modified nucleic acid residues, non-nucleotide residues or a combination thereof. In some embodiments, the 5′-HPA comprises: i) a first segment comprising at least one primer-specific sequence; ii) a second segment comprising a sequence substantially complementary to the first segment, iii) a loop connecting the first segment and the second segment; iv) free 3′-end and 5′-end which are not connected to the loop; wherein the free 3′ end of the first segment comprises 3′-OH; and the 5′ end of the second segment comprises an overhang of 5 or 6 randomized nucleotide residues and 5′-OH; and wherein the second segment comprises 2 or more RNA residues in its stem region located at least 9 nucleotides distance from the free 5′ end of the second segment. In some embodiments, the method comprises ligating the 3′-hairpin adapter (3′-HPA) described herein to the 3′ ends of the 5′-HPA-ssDNA ligation products to produce 5′-HPA-ssDNA-3′-HPA ligation products; wherein the 3′-HPA is comprised of a plurality of residues selected from: nucleic acid residues (DNA and RNA), modified nucleic acid residues, non-nucleotide residues or a combination thereof. In some embodiments, the 3′-HPA comprises: i) a first segment comprising at least one primer primer-specific sequence; ii) a second segment comprising a sequence substantially complementary to the first segment, iii) a loop connecting the first segment and the second segment; iv) free 5′-end and 3′-end which are not connected to the loop; wherein the free 5′ end of the first segment comprises 5′-p; and 3′ end of the second segment comprises an overhang of 5 or 6 randomized nucleotide residues and 3′-end blocking group that prevents its ligation and/or extension by a polymerase; and wherein the second segment comprises 2 or more RNA residues in its stem region. In some embodiments, after ligation to 3′ end of sample ssDNA, the remaining unligated 3′-HPA may be blocked (or inactivated) by ligating with a 3′-BON. In some other embodiments, after ligation to 5′ end of sample ssDNA, the remaining unligated 5′-HPA may be blocked (or inactivated) by ligating with a 5′-BON. In some embodiments, the method comprises cleaving the RNA residues in both 5′-HPAs and 3′-HPAs by treatment with an RNase H to produce cleaved 5′-HPA-ssDNA-3′-HPA ligation products, wherein the cleaving makes the first segments of 5′-HPA and 3′-HPA more accessible for hybridization with primers. In some embodiments, the method comprises performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification. In some embodiments, the method comprises amplifying the cleaved 5′-HPA-ssDNA-3′-HPA ligation products by a PCR using a pair of the first and the second sequencing primers to produce (amplified) sequencing library. In some embodiments, the first sequencing primer is at least partially complementary at its 3′ end to the 3′-HPA segment of the cleaved 5′-HPA-ssDNA-3′-HPA ligation products; and/or ii) the second sequencing primer is at least partially corresponding at its 3′ end to the 5′-HPA segments of the cleaved 5′-HPA-ssDNA-3′-HPA ligation products. In some embodiments, the methods comprise performing a post-amplification clean-up to purify the (amplified) sequencing library from non-extended primers and other components of extension and/or amplification reactions to produce purified sequencing libraries that are ready for sequencing.

In yet another embodiment, the method for preparing a sequencing library may comprise ligating a 5′-hairpin adapter (5′-HPA) described herein to the 5′-ends of the plurality of sample single-stranded DNA (sample ssDNAs) to produce a mixture comprising a plurality of 5′-HPA-ssDNA ligation products, wherein the 5′-HPA is comprised of a plurality of residues selected from: nucleic acid residues (DNA and RNA), modified nucleic acid residues, non-nucleotide residues or a combination thereof. In some embodiments, the 5′-HPA comprises: i) a first segment comprising at least one primer primer-specific sequence; ii) a second segment comprising a sequence substantially complementary to the first segment, iii) a loop connecting the first segment and the second segment; and/or iv) free 3′-end and 5′-end which are not connected to the loop; wherein the free 3′ end of the first segment comprises 3′-OH; and the 5′ end of the second segment comprises an overhang of 5 or 6 randomized nucleotide residues and 5′-OH; and wherein the second segment comprises 2 or more RNA residues in its stem region located at least 9 nucleotides distance from the free 5′ end of the second segment. In some embodiments, the method comprises ligating the 3′-hairpin adapter (3′-HPA) described herein to the 3′ ends of the 5′-HPA-ssDNA ligation products in the presence of polynucleotide kinase (PNK) to produce 5′-HPA-ssDNA-3′-HPA ligation products; wherein the 3′-HPA is comprised of a plurality of residues selected from: nucleic acid residues (DNA and RNA), modified nucleic acid residues, non-nucleotide residues or a combination thereof. In some embodiments, the 3′-HPA comprises: i) a first segment comprising at least one primer primer-specific sequence; ii) a second segment comprising a sequence substantially complementary to the first segment, iii) a loop connecting the first segment and the second segment; and/or iv) free 5′-end and 3′-end which are not connected to the loop; wherein the free 5′ end of the first segment comprises 5′-p; and 3′ end of the second segment comprises an overhang of 5 or 6 randomized nucleotide residues and 3′-end blocking group that prevents its ligation and/or extension by a polymerase; and wherein the second segment comprises 2 or more RNA residues in its stem region. In some embodiments, after ligation to 3′ end of sample ssDNA, the remaining unligated 3′-HPA may be blocked (or inactivated) by ligating with a 3′-BON. In some other embodiments, after ligation to 5′ end of sample ssDNA, the remaining unligated 5′-HPA may be blocked (or inactivated) by ligating with a 5′-BON. In some embodiments, the method comprises cleaving the RNA residues in both 5′-HPAs and 3′-HPAs by treatment with an RNase H to produce cleaved 5′-HPA-ssDNA-3′-HPA ligation products, wherein the cleaving makes the first segments of 5′-HPA and 3′-HPA more accessible for hybridization with primers. In some embodiments, the method comprises performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification. In some embodiments, the method comprises amplifying the cleaved 5′-HPA-ssDNA-3′-HPA ligation products by a PCR using a pair of the first and the second sequencing primers to produce (amplified) sequencing library. In some embodiments, the first sequencing primer is at least partially complementary at its 3′ end to the 3′-HPA segment of the cleaved 5′-HPA-ssDNA-3′-HPA ligation products. In some embodiments, the second sequencing primer is at least partially corresponding at its 3′ end to the 5′-HPA segments of the cleaved 5′-HPA-ssDNA-3′-HPA ligation products. In some embodiments, the methods comprise performing a post-amplification clean-up to purify the (amplified) sequencing library from non-extended primers and other components of extension and/or amplification reactions to produce purified sequencing libraries that are ready for sequencing.

In another embodiment, the method for preparing a sequencing library may comprise: a) ligating the 3′-HPA described herein to the 3′-ends of the plurality of sample ssDNAs to produce a mixture comprising a plurality of ssDNA-3′-HPA ligation products, wherein: i) the 3′-HPA comprises 2 or more RNA residues located at least 9 nucleotides distance from the free end of its second segment in the stem region; and ii) the 3′-HPA comprises 5 or 6 randomized nucleotide residues at its 3′-end and a 3′-end blocking group; b) ligating the 5′-HPA described herein to the 5′ ends of the ssDNA-3′-HPA ligation products to produce 5′-HPA-ssDNA-3′-HPA ligation products; wherein: i) the 5′-HPA comprises 2 or more RNA residues located at least 9 nucleotides distance from the free end of its second segment in the stem region; and. ii) the 5′-HPA overhang comprises 5 or 6 randomized nucleotide residues at its 5′-end or a 5′-end blocking group; c) cleaving the RNA residues in the 5′-HPAs and 3′-HPAs by treatment with RNase H to produce cleaved 5′-HPA-ssDNA-3′-HPA ligation products, wherein the cleaving makes the first segments of 5′-HPA and 3′-HPA more accessible for hybridization with primers; d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification; e) amplifying the cleaved 5′-HPA-ssDNA-3′-HPA ligation products by a PCR using a pair of the first and the second sequencing primers to produce (amplified) sequencing library, wherein: i) the first sequencing primer is at least partially complementary to the 3′-HPA segment of the cleaved 5′-HPA-ssDNA-3′-HPA ligation products; and ii) the second sequencing primer is at least partially corresponding to the 5′-HPA segments of the cleaved 5′-HPA-ssDNA-3′-HPA ligation products; f) performing a post-amplification clean-up to purify the amplified sequencing library from non-extended primers and other components of extension and/or amplification reactions to produce purified sequencing libraries that are ready for sequencing. Examples of such approaches are schematically shown in FIG. 2A.

In another embodiment, the method for preparing a sequencing library may comprise: a) ligating the 5′-HPA described herein to the 5′-ends of the plurality of sample ssDNAs to produce a mixture comprising a plurality of 5′-HPA-ssDNA ligation products, wherein: i) the 5′-HPA comprises 2 or more RNA residues located at least 9 nucleotides distance from the free end of its second segment in the stem region; and ii) the 5′-HPA comprises 5 or 6 randomized nucleotide residues at its 5′-end; b) ligating the 3′-HPA described herein to the 3′ ends of the 5′-HPA-ssDNA ligation products to produce 5′-IPA-ssDNA-3′-HPA ligation products; wherein: i) the 3′-HPA comprises 2 or more RNA residues located at least 9 nucleotides distance from the free end of its second segment in the stem region; and. ii) the 3′-HPA overhang comprises 5 or 6 randomized nucleotide residues at its 3′-end and a 3′-end blocking group; c) cleaving the RNA residues in the 5′-HPAs and 3′-HPAs by treatment with RNase H to produce cleaved 5′-HPA-ssDNA-3′-HPA ligation products, wherein the cleaving makes the first segments of 5′-HPA and 3′-HPA more accessible for hybridization with primers; d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification; e) amplifying the cleaved 5′-HPA-ssDNA-3′-HPA ligation products by a PCR using a pair of the first and the second sequencing primers to produce (amplified) sequencing library, wherein: i) the first sequencing primer is at least partially complementary to the 3′-HPA segment of the cleaved 5′-HPA-ssDNA-3′-HPA ligation products; and ii) the second sequencing primer is at least partially corresponding to the 5′-HPA segments of the cleaved 5′-HPA-ssDNA-3′-HPA ligation products; f) performing a post-amplification clean-up to purify the amplified sequencing library from non-extended primers and other components of extension and/or amplification reactions to produce purified sequencing libraries that are ready for sequencing. Examples of such approaches are schematically shown in FIG. 2B.

In yet another embodiments, ligating the 5′-HPA described herein to the 5′-ends of the plurality of sample ssDNAs to produce a mixture comprising a plurality of 5′-HPA-ssDNA ligation products, wherein: i) the 5′-HPA comprises 2 or more RNA residues located at least 9 nucleotides distance from the free end of its second segment in the stem region; and ii) the 5′-HPA comprises 5 or 6 randomized nucleotide residues at its 5′-end; b) ligating the 3′-HPA described herein to the 3′ ends of the 5′-HPA-ssDNA ligation products in the presence of polynucleotide kinase (PNK) to produce 5′-HPA-ssDNA-3′-HPA ligation products; wherein: i) the 3′-HPA comprises 2 or more RNA residues located at least 9 nucleotides distance from the free end of its second segment in the stem region; and ii) the 3′-HPA overhang comprises 5 or 6 randomized nucleotide residues at its 3′-end and a 3′-end blocking group; c) cleaving the RNA residues in the 5′-HPAs and 3′-HPAs by treatment with RNase H to produce cleaved 5′-HPA-ssDNA-3′-HPA ligation products, wherein the cleaving makes the first segments of 5′-HPA and 3′-HPA more accessible for hybridization with primers; d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification; e) amplifying the cleaved 5′-HPA-ssDNA-3′-HPA ligation products by a PCR using a pair of the first and the second sequencing primers to produce (amplified) sequencing library, wherein: i) the first sequencing primer is at least partially complementary to the 3′-HPA segment of the cleaved 5′-HPA-ssDNA-3′-HPA ligation products; and ii) the second sequencing primer is at least partially corresponding to the 5′-HPA segments of the cleaved 5′-HPA-ssDNA-3′-HPA ligation products; f) performing a post-amplification clean-up to purify the amplified sequencing library from non-extended primers and other components of extension and/or amplification reactions to produce purified sequencing libraries that are ready for sequencing. Examples of such approaches are schematically shown in FIG. 2C.

In yet another embodiment, the method for preparing a sequencing library may comprise: a) ligating a 5′-hairpin adapter (5′-HPA) described herein to the 5′-ends of the plurality of sample single-stranded DNA (sample ssDNAs) to produce a mixture comprising a plurality of 5′-HPA-ssDNA ligation products, wherein the 5′-HPA is comprised of a plurality of residues selected from: nucleic acid residues (DNA and RNA), modified nucleic acid residues, non-nucleotide residues or a combination thereof; wherein the 5′-HPA comprises: i) a first segment comprising at least one primer-specific sequence; ii) a second segment comprising a sequence substantially complementary to the first segment, iii) a loop connecting the first segment and the second segment; iv) free 3′-end and 5′-end which are not connected to the loop; wherein the free 3′ end of the first segment comprises 3′-OH; and the 5′ end of the second segment comprises an overhang of 5 or 6 randomized nucleotide residues and 5′-OH; and wherein the second segment comprises 2 or more RNA residues in its stem region located at least 9 nucleotides distance from the free 5′ end of the second segment; and b) ligating the 3′-hairpin adapter (3′-HPA) described herein to the 3′ ends of the 5′-HPA-ssDNA ligation products to produce 5′-HPA-ssDNA-3′-HPA ligation products; wherein the 3′-HPA is comprised of a plurality of residues selected from: nucleic acid residues (DNA and RNA), modified nucleic acid residues, non-nucleotide residues or a combination thereof, wherein the 3′-HPA comprises: i) a first segment comprising at least one primer primer-specific sequence; ii) a second segment comprising a sequence substantially complementary to the first segment, iii) a loop connecting the first segment and the second segment; iv) free 5′-end and 3′-end which are not connected to the loop; wherein the free 5′ end of the first segment comprises 5′-p; and 3′ end of the second segment comprises an overhang of 5 or 6 randomized nucleotide residues and 3′-end blocking group that prevents its ligation and/or extension by a polymerase; and wherein the second segment comprises 2 or more RNA residues in its stem region; and c) cleaving the RNA residues in both 5′-HPAs and 3′-HPAs by treatment with an RNase H to produce cleaved 5′-HPA-ssDNA-3′-HPA ligation products, wherein the cleaving makes the first segments of 5′-HPA and 3′-HPA more accessible for hybridization with primers; d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification; e) amplifying the cleaved 5′-HPA-ssDNA-3′-HPA ligation products by a PCR using a pair of the first and the second sequencing primers to produce (amplified) sequencing library, wherein: i) the first sequencing primer is at least partially complementary at its 3′ end to the 3′-HPA segment of the cleaved 5′-HPA-ssDNA-3′-HPA ligation products; and ii) the second sequencing primer is at least partially corresponding at its 3′ end to the 5′-HPA segments of the cleaved 5′-HPA-ssDNA-3′-HPA ligation products; f) performing a post-amplification clean-up to purify the (amplified) sequencing library from non-extended primers and other components of extension and/or amplification reactions to produce purified sequencing libraries that are ready for sequencing. Examples of such approaches are schematically shown in FIG. 2B.

In yet another embodiment, the method for preparing a sequencing library may comprise: a) ligating a 5′-hairpin adapter (5′-HPA) described herein to the 5′-ends of the plurality of sample single-stranded DNA (sample ssDNAs) to produce a mixture comprising a plurality of 5′-HPA-ssDNA ligation products, wherein the 5′-HPA is comprised of a plurality of residues selected from: nucleic acid residues (DNA and RNA), modified nucleic acid residues, non-nucleotide residues or a combination thereof; wherein the 5′-HPA comprises: i) a first segment comprising at least one primer primer-specific sequence; ii) a second segment comprising a sequence substantially complementary to the first segment, iii) a loop connecting the first segment and the second segment; iv) free 3′-end and 5′-end which are not connected to the loop; wherein the free 3′ end of the first segment comprises 3′-OH; and the 5′ end of the second segment comprises an overhang of 5 or 6 randomized nucleotide residues and 5′-OH; and wherein the second segment comprises 2 or more RNA residues in its stem region located at least 9 nucleotides distance from the free 5′ end of the second segment; and b) ligating the 3′-hairpin adapter (3′-HPA) described herein to the 3′ ends of the 5′-HPA-ssDNA ligation products in the presence of polynucleotide kinase (PNK) to produce 5′-HPA-ssDNA-3′-HPA ligation products; wherein the 3′-HPA is comprised of a plurality of residues selected from: nucleic acid residues (DNA and RNA), modified nucleic acid residues, non-nucleotide residues or a combination thereof, wherein the 3′-HPA comprises: i) a first segment comprising at least one primer primer-specific sequence; ii) a second segment comprising a sequence substantially complementary to the first segment, iii) a loop connecting the first segment and the second segment; iv) free 5′-end and 3′-end which are not connected to the loop; wherein the free 5′ end of the first segment comprises 5′-p; and 3′ end of the second segment comprises an overhang of 5 or 6 randomized nucleotide residues and 3′-end blocking group that prevents its ligation and/or extension by a polymerase; and wherein the second segment comprises 2 or more RNA residues in its stem region; and c) cleaving the RNA residues in both 5′-HPAs and 3′-HPAs by treatment with an RNase H to produce cleaved 5′-HPA-ssDNA-3′-HPA ligation products, wherein the cleaving makes the first segments of 5′-HPA and 3′-HPA more accessible for hybridization with primers; d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification; e) amplifying the cleaved 5′-HPA-ssDNA-3′-HPA ligation products by a PCR using a pair of the first and the second sequencing primers to produce (amplified) sequencing library, wherein: i) the first sequencing primer is at least partially complementary at its 3′ end to the 3′-HPA segment of the cleaved 5′-HPA-ssDNA-3′-HPA ligation products; and ii) the second sequencing primer is at least partially corresponding at its 3′ end to the 5′-HPA segments of the cleaved 5′-HPA-ssDNA-3′-HPA ligation products; f) performing a post-amplification clean-up to purify the (amplified) sequencing library from non-extended primers and other components of extension and/or amplification reactions to produce purified sequencing libraries that are ready for sequencing. Examples of such approaches are schematically shown in FIG. 2C.

The sequences of HPAs and sequencing primers may be compatible with current high-throughput sequencing technologies selected from: second-generation or next-generation sequencing (NGS) and third-generation or direct, single-molecule sequencing. These technologies may include (but not limited to) Solexa (Illumina), Ion Torrent (Thermo Fisher), G4 (Singular Genomics), Loop Genomics (Element Biosciences), CMOS (Genapsys); HiFi (Pacific Biosciences) and Oxford Nanopore sequencing.

In some embodiments, sequence-by-synthesis is performed. A sample DNA undergoes one round of amplification, after which the sequence is detected and analyzed. In some embodiments, sequencing—by synthesis involves fluorescently tagged dNTPs. The fluorophore acts as a reversible blocking group. After each round of synthesis, the blocking group is removed and another nucleotide is added. In some embodiments, sequencing-by-synthesis releases only a single species of dNTP each round and detects the release of a hydrogen ion released when the nucleotide is incorporated into the strand. In some embodiments, single-molecule real time sequencing is performed. In single-molecule real time sequencing, single DNA molecules are immobilised at the bottom of these wells whilst DNA polymerase incorporates fluorescently labelled nucleotides.

In some embodiments, nanopore sequencing is performed. In nanopore sequencing, an ionic current is passed through nanopores and the changes in electrical charge is measured as the nucleotides of the sample DNA pass through the pores. These sequencing primers may be suitable for single-read and/or paired-end sequencing. These sequencing primers may comprise molecular codes selected from: bar-codes, Zip-codes, sequencing indexes, unique molecular identifiers (UMI or UID) or combination thereof.

The methods described herein include a kit for preparing a sample ssDNA sequencing library comprising a 3′-HPA, a 5′-HPA, a ligase, an RNA cleaving agent, buffers, and optional components selected from: PNK, a polymerase, a 5′-BON, 3′-BON, and clean-up beads or combinations thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Non-limiting examples of “sample” include any material from which nucleic acids can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the term “free end” refers to a 5′ end or a 3′ end of a nucleic acid, wherein the 5′ end or the 3′ end is not connected to a loop.

The section headings used herein are for organizational purposes only and are not to be construed as limiting the subject matter described.

### EMBODIMENTS

Disclosed herein, in further embodiments are:
1. A composition suitable for preparation of a single-stranded DNA (ssDNA) sequencing library comprising:
   (a) a first hairpin adapter (HPA1) and a second hairpin adapter (HPA2) comprised of a plurality of residues selected from: nucleic acid residues, modified nucleic acid residues, non-nucleotide residues or a combination thereof, wherein the HPA1 and the HPA2 comprise:
      i) a first segment comprising at least one primer-specific or probe-specific sequence;
      ii) a second segment comprising a sequence substantially complementary to the first segment,
      iii) a loop connecting the first segment and the second segment;
         wherein a free end of the second segment comprises an overhang of at least one residue, wherein the second segment comprises a cleavable moiety located at least 9 nucleotides distance from the free end of the second segment; and
         wherein a free end of the first segment of HPA1 is capable of ligating to a first end of a sample ssDNA, and a free end of the first segment of HPA2 is capable of ligating to the other segment of the sample ssDNA.
2. The composition of embodiment 1, wherein the HPA1 and the HPA2 consist of:
   i) a first segment comprising at least one primer-specific or probe-specific sequence;
   ii) a second segment comprising a sequence substantially complementary to the first segment,
   iii) a loop connecting the first segment and the second segment;
      wherein a free end of the second segment comprises an overhang of at least one residue, wherein the second segment comprises a cleavable moiety located at least 9 nucleotides distance from the free end of the second segment; and
      wherein a free end of the first segment of HPA1 is capable of ligating to a first end of a sample ssDNA, and a free end of the first segment of HPA2 is capable of ligating to the other segment of the sample ssDNA.
3. The composition of embodiment 1 or 2, further comprising a blocking oligonucleotide (BON) comprising:
   i) a free end that enables ligation of the BON with any HPA1 remaining unligated after the the ligation of the HPA1 to the ssDNA to reduce ligation between the HPA1 and the HPA2; and
   ii) a second end.
4. The composition of embodiment 1 or 2, wherein the free end of the second segment of the HPA1, the free end of the second segment of the HPA2, or the free end of the second segment of the HPA1 and the free end of the second segment of the HPA2 comprise a blocking group that prevents its ligation by a ligase and/or extension by a polymerase.
5. The composition of embodiment 1 or 2, wherein the second end of the BON comprises a blocking group that disallows its ligation and/or extension by a polymerase.
6. The composition of embodiment 1 or 2, wherein the primer-specific and/or probe-specific sites are at least partially complementary or corresponding to sequences of primers and probes selected from: amplification primers, sequencing primers, detection primers, detection probes, hybridization probes, capture probes or combination thereof.
7. The composition of embodiment 1 or 2, wherein the first segment and the second segment form a double-stranded stem structure with at least 1, 2, 3, or more mismatches.
8. The composition of embodiment 1, wherein the double-stranded stem structure comprises 3 or more nucleotide base pairs.
9. The composition) of embodiment 1 or 2, wherein the loop comprises 1 or more residues selected from: nucleotide, modified nucleotide and non-nucleotide linkers or moieties.
10. The composition of embodiment 1 or 2, wherein the cleavable residues or moieties are selected from: RNA, deoxyuridine (dU), deoxyinosine (dI); internucleotide disulfide (S—S) linker(s) and internucleotide, bridging or non-bridging phosphorothioate (PS) linkage(s).
11. The composition of embodiment 1 or 2, wherein the overhang comprises one or more randomized (or degenerate) nucleotide residues (N) or defined nucleic acid and/or modified nucleic acid residues or combinations thereof, allowing simultaneous ligation with any sample ssDNA regardless of its end sequences.
12. The composition of embodiment 1 or 2, wherein the overhang comprises from 1 to 12 randomized residues selected from: nucleotide residues, modified nucleotide residues, or a combination thereof.
13. The composition of embodiment 1 or 2, wherein the free end of HPA1 is selected from: 5′-hydroxyl (5′-OH), 5′-phosphate (5′-p), 3′-hydroxyl (3′-OH) or combination thereof.
14. The composition of embodiment 1, wherein the free end of HPA2 is selected from: 5′-hydroxyl (5′-OH), 5′-phosphate (5′-p), 3′-hydroxyl (3′-OH) or combination thereof.
15. The composition of any one of embodiments 1 to 11, wherein the HPA1 or the HPA2 is a 3′-HPA that can be ligated to the 3′-end of a sample DNA.
16. The composition of any one of embodiments 1 to 11, wherein the HPA1 or the HPA2 is a 5′-HPA that can be ligated to the 5′-end of a sample DNA.
17. The composition of embodiment 1 or 2, wherein the BON comprises a structure selected from: single-stranded, double-stranded, hairpin, or combination thereof.
18. The composition of embodiment 1 or 2, wherein the BON comprises nucleotide compositions selected from: DNA, RNA; randomized (or degenerate) nucleotide residues (N); defined nucleic acid residues; modified nucleic acid residues; or a combination thereof.
19. The composition of embodiment 1 or 2, wherein the BON comprises from 1 to 12 randomized nucleotide residues.
20. The composition of embodiment 1 or 2, wherein the free end of the BON or the second end of the BON comprises sequences selected from 4 to 6 random nucleotides.
21. The composition of embodiment 1 or 2, wherein the free end of the BON or the second end of the BON are selected from: a 5′-OH, 5′-p and 3′-OH.
22. The composition of embodiment 1 or 2, wherein the blocking group is selected from: 5′-OH, 5′-amino, 5′-O-methyl and 5′-biotin linker (5′-end blocking groups); and 3′-p, dideoxynucleoside (e.g., 3′-ddC), 3-inverted dT (idT), 3′-C3 spacer, 3′-amino, and 3′-biotin linker (3′-end “blocking group”.
23. A method for preparing a sequencing library for a plurality of sample ssDNAs, comprising:
   a) ligating the HPA1 of embodiment 1 to the first end of a plurality of sample ssDNAs to produce a mixture comprising a plurality of HPA1-ssDNA or ssDNA-HPA1 ligation products and unligated HPA1;
   b) ligating any HPA1 remaining unligated after step a) with the BON of embodiment 1 to produce HPA1-BON ligation product(s) that prevent its ligation to HPA 2 (adapter dimers formation) in a downstream ligation reaction;
   c) ligating the HPA2 of embodiment 1 to the second end of the HPA1-ssDNA ligation products to produce 5′-HPA-DNA-3′-HPA (5′-HPA1-ssDNA-HPA2-3′ or 5′-HPA2-ssDNA-HPA1-3′) ligation products, wherein the sample ssDNA is positioned between the HPA1 and HPA2;
   d) cleaving the of the HPA(s) to produce cleaved 5′-HPA-DNA-3′-HPA ligation products, wherein the cleaving converts the HPAs to opened forms that are more accessible for hybridization with primer(s) and/or probe(s); and
   e) hybridizing a primer comprising a sequence at least partially complementary to the 3′-HPA(s) segment of the cleaved 5′-HPA-ssDNA-3′-HPA products; and extending the primer with a polymerase to produce a plurality of the extended products (amplification templates) representing a sequencing library.
24. The method of embodiment 23, further comprising removing components of upstream reactions that may inhibit downstream amplification reactions while preserving 5′-HPA-ssDNA-3′-HPA ligation products comprising ultrashort ssDNA fragments.
25. The method of embodiment 23, further comprising amplifying the plurality of amplification templates to produce an amplified sequencing library.
26. The method of embodiment 23, further comprising performing a post-amplification clean-up to purify the amplified sequencing library from non-extended primers and other components of extension and/or amplification reactions.
27. The method of embodiment 23, wherein the sample ssDNAs are naturally occurring and/or synthetic DNA molecules selected from: single-stranded DNAs; fragmented single-stranded DNAs; denatured double-stranded DNAs; denatured fragmented double-stranded DNAs.
28. The method of embodiment 24, wherein the sample ssDNAs are short ssDNA fragments of 120 or fewer nucleotides in length.
29. The method of embodiment 24, wherein the sample ssDNAs are ultrashort ssDNA fragments in the range of 20 to 80 nucleotides in length.
30. The method of embodiment 24, wherein the sample ssDNAs are selected from: circulating tumor DNA, circulating microbial DNA, circulating bacterial DNA, circulating viral DNA, circulating mitochondrial DNA, circulating genomic DNA, circulating cell-free DNA (cfDNA) from a biofluid (liquid biopsy); DNA from formalin-fixed, paraffin-embedded (FFPE) tissue samples; highly degraded DNA from ancient organisms or forensic biological samples.
31. The method of embodiment 30, wherein the biofluid is selected from: whole blood, plasma, serum, saliva, and urine.
32. The method of embodiment 30, wherein the sample ssDNA comprises isolated total nucleic acids including both DNA and RNA.
33. The method of embodiment 30, wherein the sample ssDNA comprises isolated total DNA.
34. The method of embodiment 33, wherein the sample ssDNA comprises isolated target DNAs.
35. The method of embodiment 34, wherein the target ssDNAs are isolated by hybridization with target-specific oligonucleotide probes, wherein the hybridization is performed either in solution followed by attachment of target-probe complexes to a solid support, or on a solid phase comprising target-specific probes immobilized on a solid support.
36. The method of embodiments 35, wherein the target ssDNAs are captured directly from a biofluid or a lysed biofluid.
37. The method of embodiment 24, wherein the plurality of sample ssDNAs comprise DNA ends selected from 5′-p, 3′-OH, 5′-OH and 3′-p or combinations thereof.
38. The method of embodiment 37, wherein the sample ssDNA is repaired to convert 5′-OH and/or 3′-p ends to 5′-p and 3′-OH forms, before or simultaneous with the ligating of HPAs.
39. The method of embodiment 37, wherein the conversion is performed by a polynucleotide kinase.
40. The method of embodiment 37, wherein the ligating is performed without “repair” of ends of the sample DNA.
41. The method of embodiment 23, wherein the HPA1 is a 3′-HPA and the HPA2 is a 5′-HPA.
42. The method of embodiment 41, wherein the 3′-HPA comprises a 5′-p end and its 3′-overhang comprises a 3′-end blocking group.
43. The methods of embodiment 42, wherein the remaining unligated 3′-HPA is blocked by ligating with a 3′-blocking oligonucleotide (3′-BON) comprising a 5′-p and a 3′-end-blocking group.
44. The method of embodiment 23, wherein the HPA1 is the 5′-HPA and the HPA2 is the 3′-HPA,
45. The method of embodiment 44, wherein the 5′-HPA(s) comprises a 3′-OH and a 5′-end overhang comprising a 5′-OH or a 5′-end blocking group.
46. The method of embodiment 45, wherein the remaining unligated 5′-HPA is blocked by ligating with a 5′-blocking oligonucleotide (5′-BON) comprising a 5′-p and a 3′-end-blocking group.
47. The method of embodiment 23, wherein the HPA2 is taken in molar excess over the HPA1.
48. The method of embodiment 23, wherein the BONs are in molar excess over the HPA1 during the blocking step.
49. The method of embodiment 23, wherein the ligating is splint-dependent (or template-dependent) ligation between the sample DNAs, the HPA1, the HPA2 and/or the BON and a ligation step performed by a ligase.
50. The method of embodiment 49, wherein the ligase is selected from: T4 DNA ligase, T3 DNA ligase, T7 DNA ligase, PBCV1 DNA ligase, E. coli DNA ligase, human DNA ligase III, and SplintR® Ligase.
51. The method of embodiment 23, wherein the HPA1 or the HPA1 comprise one or more RNA residues that can be cleaved by an RNA cleaving agent selected from: a ribonuclease, hydroxyl anions, and metal cations.
52. The method of embodiment 51, wherein the ribonuclease is selected from the RNase H family.
53. The method of embodiments 23, wherein the use of a RNA cleaving agent for the HPA cleavage may simultaneously cleave any RNA present in the sample ssDNA to prevent the incorporation of RNA sequences into the ssDNA sequencing library.
54. A method for preparing a sequencing library from a plurality of sample ssDNAs, comprising:
   a) ligating the 3′-HPA of embodiment 14 to the 3′-ends of the plurality of sample ssDNAs to produce a mixture comprising a plurality of ssDNA-3′-HPA ligation products and remaining unligated 3′-HPAs, wherein:
      i) the 3′-HPA comprises 2 or more RNA residues in the second segment in the stem region; and
      ii) the overhangs located on the 3′-ends of the 3′-HPA comprise 5 or 6 randomized nucleotide residues and a 3′-end blocking group of embodiment 16;
   b) ligating the remaining unligated 3′-HPA with a pool of 3′-BONs of embodiment 34 containing 5 or 6 randomized RNA nucleotides to produce 3′-BON-3′-HPA ligation product (blocked 3′-HPA);
   c) ligating the 5′-HPA of embodiment 15 to the 5′ ends of the ssDNA-3′-HPA ligation products to produce 5′-HPA-ssDNA-3′-HPA ligation products; wherein:
      i) the 5′-HPA comprise 2 or more RNA residues in the second segment in the stem region; and
      ii) the overhangs located on the 5′-end of 5′-HPA comprise 5 or 6 randomized nucleotide residues at the 5′-end of the overhang;
   d) cleaving the RNA residues in both 5′-HPAs and 3′-HPAs by treatment with an RNA cleaving agent to produce cleaved 5′-HPA-ssDNA-3′-HPA products;
   e) removing components of upstream reactions that may inhibit downstream amplification reactions while preserving 5′-HPA-DNA-3′-HPA ligation products comprising ultrashort ssDNA fragments (“pre-PCR-cleanup);
   f) preparing amplified sequencing library by PCR amplification of a plurality of the cleaved 5′-HPA-ssDNA-3′-HPA products using a pair of sequencing primers that are specific (complementary or corresponding) to remaining adapter first segments in the cleaved 5′-HPA-ssDNA-3′-HPA products;
   g) purifying the amplified sequencing library from non-extended primers and other components of extension and/or amplification reactions (post-PCR clean-up) to produce purified sequencing libraries that are ready for sequencing.
55. A method for preparing a sequencing library from a plurality of sample ssDNAs, comprising:
   a) simultaneously ligating a plurality of 3′ Hairpin Adapters (3′-HPAs) and 5′ Hairpin Adapters (5′-HPAs) with sample ssDNAs to produce a mixture comprising a plurality of 5′-HPA-ssDNA-3′-HPA ligation products in which the ssDNA is inserted between the 5′-HPA and the 3′-HPA, wherein:
      i) the 3′-HPAs comprise 2 or more RNA residues in the second segment in the stem region; and
      ii) the overhang located on the 3′-ends of the 3′-HPAs comprise 5 or 6 randomized nucleotide residues and a 3′-end blocking group;
      iii) the 5′-HPAs comprise 2 or more RNA residues in the second segment in the stem region; and
      iv) the overhangs located on the 5′-end of 5′-HPAs comprise 5 or 6 randomized nucleotide residues at 5′-end of the overhang;
   b) cleaving the RNA residues in both 5′-HPAs and 3′-HPAs by a treatment with an RNA cleaving agent to produce a cleaved 5′-HPA-ssDNA-3′-HPA products;
   c) removing components of upstream reactions that may inhibit downstream amplification reactions and depleting adapter dimers while preserving the cleaved 5′-HPA-DNA-3′-HPA ligation products comprising ultrashort ssDNA fragments;
   d) preparing an amplified sequencing library by PCR amplification of a plurality of the cleaved 5′-HPA-ssDNA-3′-HPA products using a pair of sequencing primers that are specific (complementary or corresponding) to remaining adapter first segments in the cleaved 5′-HPA-ssDNA-3′-HPA products;
   e) purifying the amplified sequencing library from non-extended primers and other components of extension and/or amplification reactions (post-PCR clean-up) to produce purified sequencing libraries that are ready for sequencing.
56. A method for preparing a sequencing library from a plurality of sample ssDNAs, comprising:
   a) ligating a 3′-HPA to the 3′-ends of the plurality of sample ssDNAs to produce a mixture comprising a plurality of ssDNA-3′-HPA ligation products and remaining unligated 3′-HPAs, wherein the 3′-HPA is comprised of a plurality of residues selected from: nucleic acid residues, modified nucleic acid residues, non-nucleotide residues or a combination thereof; wherein the 3′-HPA comprises:
      i) a first segment comprising at least one primer specific or probe-specific sequence;
      ii) a second segment comprising a sequence substantially complementary to the first segment,
      iii) a loop connecting the first segment and the second segment;
   wherein a free end of the second segment comprises an overhang of 5 or 6 randomized nucleotide residues at the 3′ end of the 3′-HPA, wherein the free end of the second segment comprises a blocking group, wherein the second segment comprises a cleavable moiety located at least 9 nucleotides distance from the free end of the second segment; and the 3′-HPA comprises 2 or more RNA residues in the second segment in the stem region; and
   b) ligating the remaining unligated 3′-HPA with a pool of 3′-BONs to produce 3′-BON-3′-HPA ligation product (blocked 3′-HPA), wherein the 3′-BONs comprise
      i) a first end containing 5 or 6 randomized RNA nucleotides; and
      ii) a second end;
   c) ligating the 5′-HPA of embodiment 15 to the 5′ ends of the ssDNA-3′-HPA ligation products to produce 5′-HPA-ssDNA-3′-HPA ligation products; wherein the 5′ HPA is comprised of a plurality of residues selected from: nucleic acid residues, modified nucleic acid residues, non-nucleotide residues or a combination thereof, wherein the 5′-HPA comprises:
      i) a first segment comprising at least one primer specific or probe-specific sequence;
      ii) a second segment comprising a sequence substantially complementary to the first segment,
      iii) a loop connecting the first segment and the second segment;
      wherein the 5′-HPA comprise 2 or more RNA residues in the second segment in the stem region; and wherein the overhangs located on the 5′-end of 5′-HPA comprise 5 or 6 randomized nucleotide residues at the 5′-end of the overhang;
   d) cleaving the RNA residues in both 5′-HPAs and 3′-HPAs by treatment with an RNA cleaving agent to produce cleaved 5′-HPA-ssDNA-3′-HPA products;
   e) removing components of upstream reactions that may inhibit downstream amplification reactions while preserving 5′-HPA-DNA-3′-HPA ligation products comprising ultrashort ssDNA fragments (“pre-PCR-cleanup);
   f) preparing amplified sequencing library by PCR amplification of a plurality of the cleaved 5′-HPA-ssDNA-3′-HPA products using a pair of sequencing primers that are specific (complementary or corresponding) to remaining adapter first segments in the cleaved 5′-HPA-ssDNA-3′-HPA products;
   g) purifying the amplified sequencing library from non-extended primers and other components of extension and/or amplification reactions (post-PCR clean-up) to produce purified sequencing libraries that are ready for sequencing.
57. The method of any one of embodiments 23 to 56, wherein sequences of the HPAs and sequencing primers are compatible with current high-throughput, sequencing technologies selected from Illumina, ThermoFisher and GenapSys.
58. The method of any one of embodiments 23 to 57, wherein the primers are sequencing primers suitable for “single-end” (a.k.a. “single-read”) and/or “paired-end” sequencing.
59. The method of any one of embodiments 23 to 58, wherein the primers are sequencing primers comprising molecular codes selected from: bar-codes, Zip-codes, sequencing indexes, unique molecular identifiers (UMI or UID) or combination thereof.
60. A kit for preparing the sample ssDNA sequencing library comprising a 3′-HPA, a 5′-HPA, a 3′-BON, a ligase, an RNA cleaving agent, and a reaction buffer.

### EXAMPLES

The following examples are included for illustrative purposes only and are not intended to limit the scope of the invention.

### Example 1. Preparation cfDNA Sequencing Libraries Using a Variant of HASL-Free-Seq Featuring 3′→5′ Sequential Adapter Ligation with the Adapter Blocking Workflow

This workflow is schematically shown in FIGS. 2A and 3B. Master mixes for multiple reactions were prepared first. Solute and reaction volumes indicated below are per reaction. All synthetic oligonucleotides were from IDT. In Step 1, ssDNA (or denatured double-stranded DNA) inputs are ligated to 3′-HPA: 5′-/5Phos/TGGAATTCTCGGGTGCCAAGGAACTrCrCrUrUrGrGrCrACCCGAGAATTCCANN NNNN/3AmMO/-3′ (SEQ ID NO. 1). Specifically, the 3′-HPA (0.25 pmole in 1 μl) is denatured for 5 min at 95° C. in a custom “Hairpin Buffer” [0.2 mM MgCl₂, 60 mM KCl, and 6 mM HEPES-KOH (pH 7.5)] then immediately chilled on ice for 5 min. The ssDNA input (1 ng) is denatured for 5 min at 95° C. in “IDTE buffer” [10 mM Tris, 0.1 mM EDTA (pH 7.5)] and then chilled on ice for 5 min. The 3′-HPA and ssDNA inputs are ligated (to produce ssDNA-3′-HPA ligation products) by incubation for 1 hour at 30° C. with 4U (1 μl) of T4 DNA Ligase (NEB) in a custom “SMGX4 buffer” [15% w/v PEG-8000, 50 mM Tris-HCl pH 7.5, 10 mM MgCl₂, 0.05 mM ATP and 10 mM DTT] in a 20-μl reaction volume. In Step 2, the remaining unligated 3′-HPA (present in excess over ssDNA input in Step 1) is blocked by ligation with the random DNA hexamer, 5′-NNNNNN-3′ (SEQ ID NO. 5) used here as 3′-end blocking oligonucleotide (3′-BON). Specifically, the 3′-BON (135 pmoles in 2 μl) is added to the reaction mixture from Step 1 (to a total volume 22 μl) and incubated for 1 hour at 30° C. In Step 3, the 5′-HPA, 5′-NNNNNNGATCGTCGGACTGTArGrArArCrUrCrUrGrAACACGTTCAGAGTTCTACAGT CCGACGATC-3′ (SEQ ID NO. 2), is ligated to the ssDNA-3′-HPA ligation products (from Step 1) to form 5′-HPA-ssDNA-3′-HPA ligation products. The 5′-HPA (0.25 pmoles in 1 μl) is denatured in Hairpin Buffer as described for 3′-HPA in Step 1 and then added to the reaction mixture from Step 3 along with 1 μl SMGX4 buffer and 1 μl nuclease-free water (HyClone) to a total volume 25 μl. The reaction mixture is incubated for 1 hour at 30° C., the T4 DNA ligase is inactivated by incubating at 70° C. for 10 min, and then the mixture is immediately placed on ice for 5 min. In Step 4, the 5′-HPA-ssDNA-3′-HPA ligation products are processed by RNase H endonuclease as follows: the reaction mixture from Step 3 is mixed with 1.5 μl nuclease-free water, 2.5 μl 10× RNase H buffer [30 mM MgCl₂, 100 mM DTT, 500 mM Tris-HCl (pH 8.3); NEB] and 1 μl (5 U) of RNase H (NEB). The final 30-μl reaction mixture is incubated for 30 min at 30° C. and then chilled to 4° C. in a thermocycler. All reactions for Steps 1 through 4 are performed in a single tube. In Step 5, the RNase H-treated 5′-HPA-ssDNA-3′-HPA ligation products from Step 4 are denatured and then amplified by PCR using the Illumina sequencing primers. Specifically, the reaction mixture from Step 4 is diluted to 60 μl by nuclease-free water and 25 μl is used for PCR without purification of the 5′-HPA-ssDNA-3′-HPA products. The PCR reactions are performed in the presence of 0.4 μM of the RP1 and indexed RPI (1-48) Illumina primers (https://support.illumina.com/sequencing/sequencing_kits/idt-truseq-DNA-rna-udi.html) in 50-μl total reaction volumes. The PCR is performed with following program per cycle: 1: 94° C. for 30 s; 2: 15-19 cycles [94° C. for 30 s→62° C. for 30 sec→70° C. for 30 sec], and 3: 70° C. for 5 min. The number of PCR cycles is determined by the amount of starting material (DNA input); e.g., 17 PCR cycles were used for 1 ng of input DNA. The PCR amplification products were purified away from the PCR primers using SPRIselect reagent (Beckman Coulter) and analyzed by an Agilent TapeStation (D100 ScreenTape assays) before sequencing. Single-read (a.k.a. single-end) sequencing with 150 nt cut-off was performed on a MiniSeq sequencer (Illumina). Sequences were aligned to the human genome (hg19), normalized to counts per million (CPM), and were plotted against cfDNA fragment lengths. Statistical analysis of the sequencing data was performed using a custom bioinformatic pipeline. Results of a study that used this protocol are presented in EXAMPLE 2.

### Example 2. Analysis of Sequencing Profiles for HASL-Free-Seq and Competitor a Sequencing Libraries Made for cfDNA Isolated from Plasma Samples Taken from a Healthy Donor and Breast Cancer Patients

Plasma samples were taken from a healthy donor and 3 different, post-treatment patients diagnosed with different sub-types and stages of breast cancer. All plasma samples were obtained by Innovative Research (https://www.innov-research.com/collections/human-plasma). cfDNA from the plasma samples were isolated using Plasma/Serum Cell-Free Circulating DNA Purification Mini Kit (Norgen Biotek). 1 ng cfDNA inputs were used for the preparation of sequencing libraries. The HASL-free-Seq libraries were prepared as described in Example 1. The market leader in ssDNA sequencing, Swift Biosciences' Accel 1S kit (Accel-NGS® 1S Plus DNA Library Kit) was selected as a benchmark method (Competitor A) for comparison purposes. Accel 1S sequencing libraries for cfDNA were prepared using the additional option described in Appendix A of the Accel 1S protocol for small fragment (≥40 bp) retention. These libraries were sequenced as described in EXAMPLE 1.

Comparison of the sequencing profiles for libraries prepared by the Accel 1S and HASL-free-Seq protocols demonstrated the significantly increased ability of HASL-free-Seq to capture ultrashort genomic DNA fragments of ≤60 nt compared to Accel 1S. This ability of HASL-free-Seq allowed robust discrimination between cfDNA samples from the healthy donors and the cancer patients by detecting increased amounts of ultrashort cfDNA fragments of 20-80 nt in the cancer samples. The breast cancer cfDNA samples showed expected variations in fragmentation profiles (lengths of fragment sequences vs. normalized sequencing read counts) of their sequencing libraries that could be associated with the different stages of the post-treatment patients.

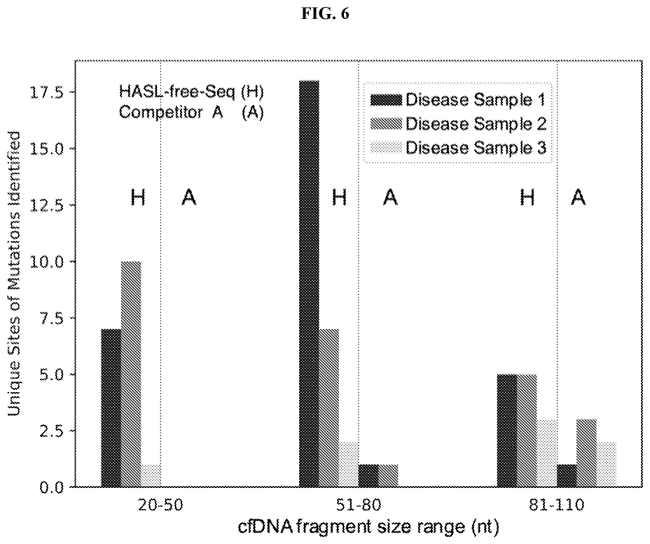

To demonstrate the importance of ultrashort cfDNAs, we analyzed these sequencing profiles to focus only on Estrogen Receptor 1 (ESR1) gene sequences and determine the numbers of unique (tumor-specific) ESR1 mutations identified in the cfDNA of breast cancer, but not in the healthy plasma samples. The detection of ESR1 mutations in cfDNA is emerging as a noninvasive biomarker of acquired resistance to endocrine therapy and is associated with hormone-resistant metastatic breast cancer. A comparison of sub-fractions within the 20-100 nt size range (FIG. 6) revealed that, for both HASL-free-Seq (H) and Accel 1S (A) methods, the most disease-specific ESR1 mutations were detected in the 51-80-nt size range, followed by the 20-50-nt range, with the fewest mutations being in the 81-100-nt range. The HASL-free-Seq approach detected significantly higher numbers of tumor-specific mutations in sequences of 20-80 nt than Accel 1S, which detected few or no mutations in ultrashort cfDNA sequences.

### Example 3. Preparation of cfDNA Sequencing Libraries Using HASL-Free-Seq Variant Featuring Sequential Adapter Ligation (without the Adapter Blocking) Protocol

### Step 1. Buffers and Equipment Preparation

1. Dilute stock 10 mM Reaction Buffer to 1.3 mM concentration and put it on ice.
2. Thaw buffers.
3. Chill aluminum cooling block on ice.
4. Pre-heat thermocycler to 95° C.

### Step 2. Adapter and DNA Sample Denaturation

1. Label two separate 0.2 ml PCR microtubes “A1” and A2″, and individual 0.2 ml PCR microtubes for each DNA sample.
2. Thaw, vortex, and spin Adapter 1 and Adapter 2.
3. Add 1 μl Adapter 1 (e.g., 3′-HPA) to tube A1 and 1 μl Adapter 2 (e.g., 5′-HPA) to tube A2 for each reaction.
4. Add 1 μl Preparation Buffer to tubes A1 and A2 for each reaction.
5. To DNA sample tubes, add desired input amount of cfDNA and adjust the volume of the sample to a final volume of 5 μl using DNA Dilution Buffer, if necessary.
6. Places all tubes in thermocycler at 95° C. for 5 minutes.
7. Pulse-spin and immediately transfer the tubes to the aluminum block chilled on ice for 5 minutes.

### Step 3. Pre-Ligation

1. Combine the following as a master mix. Mix and pulse-spin, aliquot 1 μl of the mixture into each sample tube (total reaction volume—6 μl).

| Pre-Ligation | | |
|---|---|---|
| components | Volume | |
| Pre-Ligation Buffer | 0.6 | μl |
| Nuclease-free water | 0.4 | μl |
| Total Volume | 1 | μl |

2. Pulse-spin. Incubate at 65° C. for 30 minutes.
3. After reaction is complete, spin down and immediately chill samples on an aluminum cooling block for at least 3 minutes.

### Step 4. Adapter 1 Ligation

1. Combine the following in order as a master mix on ice. Mix well and pulse-spin. (Note: Viscous solution! Mix thoroughly and pipette slowly into a reaction mixture)

| Adapter 1 Ligation components | Volume | |
|---|---|---|
| Ligation Buffer | 10 | μl |
| Adapter 1 | 1 | μl |
| 1.3 mM Reaction Buffer | 1 | μl |
| Nuclease-free water | 1 | μl |
| Ligase | 1 | μl |
| Total Volume | 14 | μl |

2. Working with prepared tubes on chilled aluminum block, distribute 14 μl of the reaction mixture to each tube with a prepared DNA sample, mix by pipetting up and down 15 times (total reaction volume—20 μl).
3. Incubate at 30° C. for 1 hour.

### Step 5. Adapter 2 Ligation

1. Combine the following in order as a master mix on ice. Mix well and pulse-spin. (Note: Viscous solution! Mix thoroughly and pipette slowly into a reaction mixture)

| Adapter 2 Ligation components | Volume | |
|---|---|---|
| Ligation Buffer | 2.5 | μl |
| 2nd Ligation Buffer | 0.25 | μl |
| 1.3 mM Reaction Buffer | 0.31 | μl |
| Nuclease-free water | 0.94 | μl |
| Adapter 2 | 1 | μl |
| Total Volume | 5 | μl |

2. Working with prepared tubes on chilled aluminum block, distribute 5 μl of the reaction mixture to each tube with the sample from Step 4 comprising ligation product [Adapter 1-DNA], mix by pipetting up and down 5 times (total reaction volume—25 μl).
3. Incubate the mixture in a thermocycler according to the following:

| Temperature | Time |
|---|---|
| 30° C. | 60 min |
| 65° C. | 10 min |

4. After reaction is complete, spin down and immediately chill samples on an aluminum cooling block for at least 3 minutes.

### Step 6. Processing of Ligated Adapters

1. Combine the following on ice, and mix.

| Hydrolysis components | Volume |
|---|---|
| Processing Buffer | 3 μl |
| Nuclease-free water | 1 μl |
| Processing Reagent | 1 μl |
| Total Volume | 5 μl |

2. Working with the ligation product from Step 5 [Adapter 1-DNA-Adapter 2] on chilled aluminum block, distribute 5 μl of the reaction mixture to each tube with the Ligated DNA sample, mix by pipetting up and down (total reaction volume—30 μl).
3. Incubate mixture at 30° C. for 30 minutes then chill on an aluminum cooling block.

### Step 7. Purification of Ligation Products

1. Make fresh 85% Ethanol (Vol=n×(0.5 ml), where n=# of samples).
2. Thoroughly vortex the SPRIselect® Magnetic Beads (Beckman Coulter) for at least 30 seconds.
3. Dilute each Ligated DNA volume with 20 μl DNA Dilution Buffer.
4. Add 75 μl of SPRIselect® beads to each sample. Pipet up and down 10 times to mix. Let samples stand at room temp for 5 minutes.
5. Place the sample tubes on a magnetic rack until the mixture clears and beads are sequestered, approximately 5 minutes.
6. Keeping the tubes on the magnet, remove the supernatant without disturbing the sequestered beads, ˜10 μl of beads in solution may be safely left behind.
7. Wash 1: add 180 μl of the 85% ethanol solution to each sample while it is still on the magnetic rack. Do not disturb the pellet.
8. Keeping the tubes on the magnet, carefully remove the ethanol without disturbing the beads in each sample, ˜10 μl of beads in solution may be safely left behind.
9. Wash 2: add another 180 μl of the 85% ethanol solution to each sample while it is still on the magnetic rack. Do not disturb the pellet.
10. Keeping the tubes on the magnet, carefully remove the ethanol without disturbing the beads in each sample, ˜10 μl of beads in solution may be safely left behind.
11. Briefly spin samples in a tabletop microcentrifuge and place them back on the magnetic rack. Wait 3 minutes then remove residual ethanol from the bottom of the tube once it is clear of beads.
12. Add 10 μl of Nuclease-free water to resuspend the beads. Pipet up and down to mix.
13. Incubate samples at room temperature for 5 minutes away from the magnet, then place tubes on the magnetic rack until the mixture is clear, approximately 3 minutes.
14. Carefully transfer 10 μl of eluate to a clean 0.2 ml PCR microtube.

Proceed immediately to next step or Stopping point: Alternatively, libraries can be stored overnight at −20° C. To restart, thaw samples on ice before proceeding to next step.

### Step 8. Amplification

1. Combine the following in order. Vortex thoroughly and pulse-spin.

| PCR component | Volume | |
|---|---|---|
| Nuclease-free water | 6.25 | μl |
| PCR Buffer | 5 | μl |
| dNTPs | 0.75 | μl |
| Forward Primer | 1 | μl |
| PCR Polymerase | 1 | μl |
| Total Volume | 14 | μl |

2. Add 14 μl of PCR reaction mixture to each 0.2 ml PCR microtube containing 10 μl of purified ligated DNA product from Step 7.
3. Add 1 μl of Reverse Primer Index to each 0.2 ml PCR microtube (total reaction 25 μl). Mix by pipetting and spin down.
   Choose unique reverse index Illumina sequencing primers for each sample.
4. Run the following PCR temperature profile. The number of cycles will be dictated by input.

| Step | Temperature ° C. | Time | |
|---|---|---|---|
| HOLD | 94 | 1 | min |
| CYCLE | 94 | 15 | s |
| (8-16 cycles) | 60 | 30 | s |
| | 70 | 30 | s |
| HOLD | 70 | 5 | min |

| | | |
|---|---|---|
| HOLD | 4 | Forever |

Proceed immediately to next step or Stopping point: Alternatively, libraries can be stored overnight at −20° C. To restart, thaw samples on ice before proceeding to next step.

### Step 9. Purification of PCR Amplicons

1. Make fresh 85% Ethanol (Vol=n×(0.5 ml) where n=# of samples).
2. Thoroughly vortex the SPRIselect® Magnetic Beads reagent for at least 30 seconds.
3. Dilute each Amplified DNA volume with 25 μl DNA Dilution Buffer.
4. Add 75 μl of SPRIselect® beads to each sample. Pipet up and down 10 times to mix. Let samples stand at room temp for 5 minutes.
5. Place the samples on a magnetic rack until the mixture clears and beads are sequestered, approximately 5 minutes.
6. Remove the supernatant without disturbing the sequestered beads, ˜10 μl of beads in solution may be safely left behind. Leave the tubes on the magnet.
7. Wash 1: add 180 μl of the 85% ethanol solution to each sample while it is still on the magnetic rack. Do not disturb the pellet.
8. Keeping the tubes on the magnet, carefully remove the ethanol without disturbing the beads in each sample, ˜10 μl of beads in solution may be safely left behind.
9. Wash 2: add another 180 μl of the 85% ethanol solution to each sample while it is still on the magnetic rack. Do not disturb the pellet.
10. Keeping the tubes on the magnet, carefully remove the ethanol without disturbing the beads in each sample, ˜10 μl of beads in solution may be safely left behind.
11. Briefly spin samples in a tabletop microcentrifuge and place them back on the magnetic rack. Wait 3 minutes then remove residual ethanol from the bottom of the tube once it is clear of beads.
12. Add 10 μl of Nuclease-free water to resuspend the beads. Pipet up and down to mix.
13. Incubate samples at room temperature for 5 minutes away from the magnet, then place tubes on the magnetic rack until the mixture is clear, approximately 3 minutes.
14. Carefully transfer 10 μl of eluate to a clean 0.2 ml PCR microtube. This material is now ready for end-point analysis pooling and sequencing. Store at −20° C.

### Example 4: Analysis of Sequencing Profiles for HASL-Free-Methyl-Seq and Competitor B Sequencing Libraries Made for cfDNA Isolated from Plasma Samples Taken from a Healthy Donor

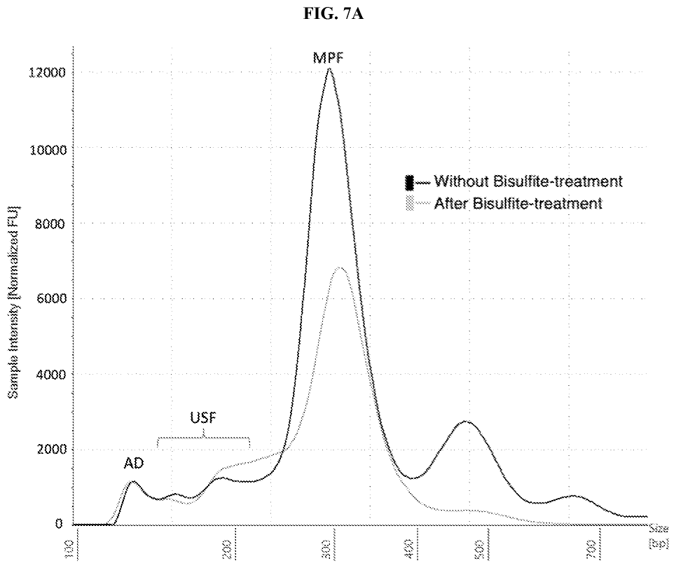

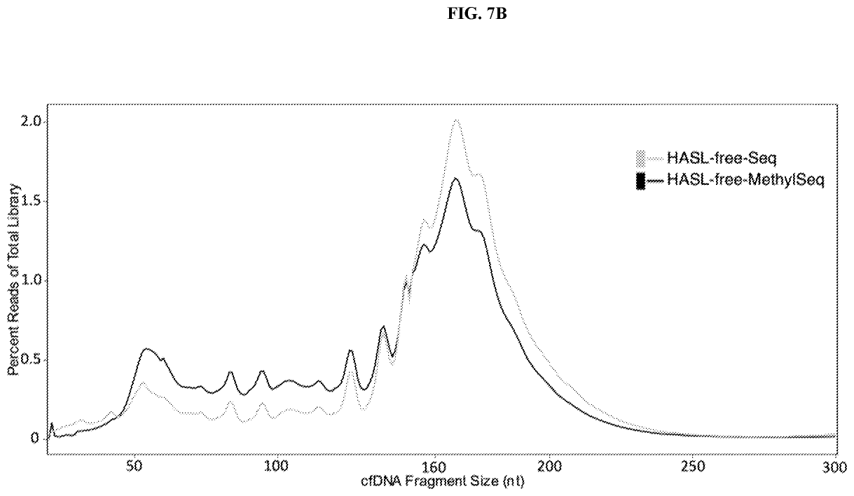

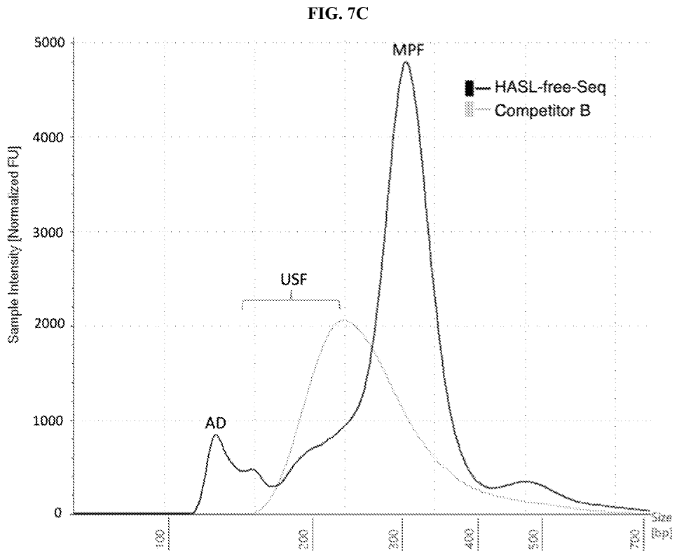

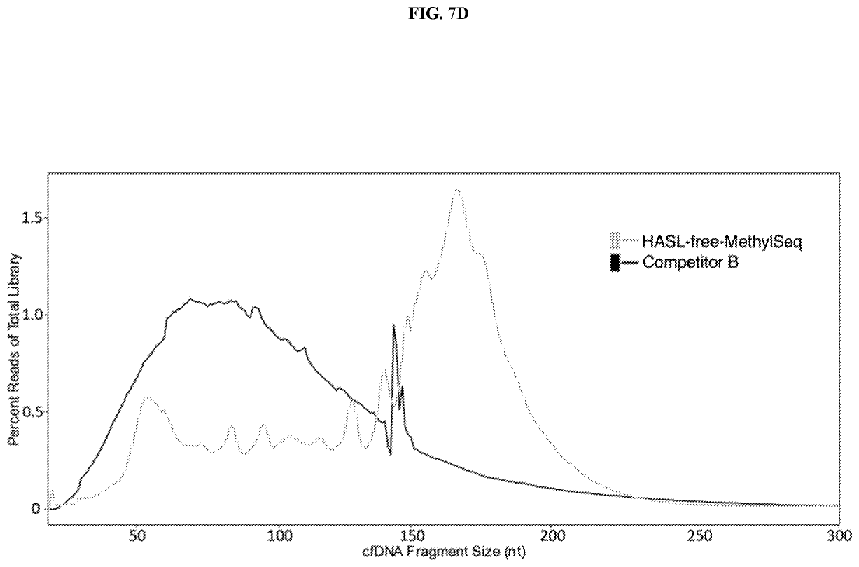

cfDNA was isolated using Plasma/Serum Cell-Free Circulating DNA Purification Mini Kit (Norgen Biotek) from the plasma samples of a healthy donor obtained by Innovative Research (https://www.innov-research.com/collections/human-plasma). 3 ng cfDNA were input for bisulfite treatment using the EZ DNA Methylation Lightning® kit (Zymo Research). The bisulfite treatment was used for identification of 5-methylcytosine and 5-hydroxymethylcytosine nucleotides in the sample cfDNA by Illumina sequencing. For preparation of the HASL-free-Methyl-Seq library, estimated (based on the bisulfite treatment DNA recovery efficiency) 0.7 ng input of bisulfite-treated cfDNA was used. These libraries were prepared featuring the 5′→3′ sequential ligation as schematically shown in FIG. 2C. A comparison of the HASL-free-Methyl-Seq protocol was made on 0.5 ng input of cfDNA that was not bisulfite-treated. Agilent TapeStation sequencing library profiles for cfDNA samples is depicted in FIG. 7A. The library prepared for the bisulfite-treated cfDNA showed higher level of DNA fragmentation (increased USF library fraction) and overall lower yields for longer library fractions in comparison to the non-treated cfDNA profile.

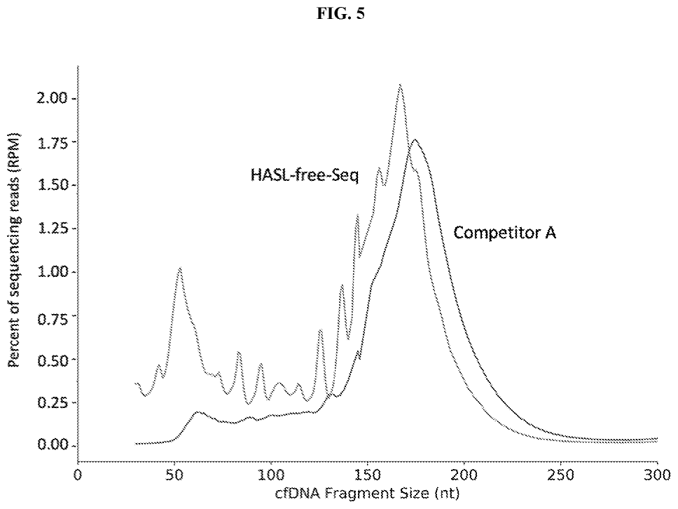

The fragmentation sequencing profiles plotted as percent of human genome (hg19) aligned sequencing reads against cfDNA fragment lengths (as described in the legend to FIG. 5) provides further evidence of increasing numbers of the ultrashort cfDNA fragments after the bisulfite treatment (FIG. 7B). The height of the peaks in the fragmentation profiles depends only on the numbers of sequencing reads for cfDNA fragments rather than their lengths, whereas in the TapeStation profiles (FIG. 7A), the height of the peaks for cfDNA fragments incorporated into sequencing libraries is proportional to the length of (or number of nucleotides in) these fragments that explains the differences between these profiles.

Competitor B (Pico Methyl-Seq™ Library Prep Kit from Zymo Research) was selected as a benchmark method for comparison with the HASL-free-Methyl-Seq protocol. After the bisulfite treatment, the same treated cfDNA inputs were used to prepare sequencing libraries using Methyl-Seq variant of HASL-free-Seq and Competitor B protocol. Agilent TapeStation sequencing library profiles are depicted in FIG. 7C. Overall, the Methyl-Seq variant of HASL-free-Seq appeared to be highly capable in capturing larger numbers and varieties of bisulfite-treated cfDNA fragments that might differ both in size and sequences. Aliquots of the same bisulfite-treated cfDNA samples (with the same level cfDNA fragmentation) were used for preparation of sequencing libraries by both methods. Sequencing profiles are depicted in FIG. 7D. Therefore, the dramatic increases of ultrashort and decreases of longer fragments of cfDNA in both TapeStation and fragmentation sequencing profiles for the Competitor B is a result of artificial shortening of the sample cfDNA and an inability of this method to incorporate full-length cfDNA fragment sequences present in the sample cfDNA into the sequencing libraries. The latter emphasize the advantage of HASL-free-Methyl-Seq in preparation of sequencing libraries for highly fragmented cfDNA that allows to capture complete sequencing information for all cfDNA fragments including their fine sawtooth-like patterns and end sequences. These features could result in higher sensitivity and specificity in detecting disease-specific alteration of cfDNA methylation patterns.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| SEQ ID | |
|---|---|
| NO | Sequence |
| 1 | tggaattctc gggtgccaag gaactccuug gcacccgaga |
| | attccannnn nn |
| 2 | nnnnnngatc gtcggactgt agaacucuga acacgttcag |
| | agttctacag tccgacgatc |
| 3 | acacgtctga actccagtca caactgugac uggagutcag |
| | acgtgtnnnn nn |
| 4 | nnnnnngtcg tgtagggaaa gagugucacg acactctttc |
| | cctacacgac |
| 5 | nnnnnn |
| 6 | cnnnnn |
| 7 | cannnn |
| 8 | tcccgtacga tgctgnnnnn n |
| 9 | tcccgtacga tgctgcnnnn n |
| 10 | tcccgtacga tgctgcannn n |
| 11 | cagcatcgta cgggacacgt cccgtacgat gctgnnnnnn |
| 12 | cagcatcgta cgggacacgt cccgtacgat gctgcnnnnn |
| 13 | cagcatcgta cgggacacgt cccgtacgat gctgcannnn |

What is claimed is:

1. A composition suitable for preparation of a single-stranded DNA (ssDNA) sequencing library comprising:
(a) a first hairpin adapter (HPA1) and a second hairpin adapter (HPA2) comprised of a plurality of residues selected from: nucleic acid residues, modified nucleic acid residues, non-nucleotide residues or a combination thereof, wherein the HPA1 and the HPA2 comprise:
i) a first segment comprising at least one primer-specific or probe-specific sequence;
ii) a second segment comprising a sequence substantially complementary to the first segment; and
iii) a loop connecting the first segment and the second segment;
wherein a free end of the second segment comprises an overhang of at least one residue,
wherein the second segment comprises a cleavable moiety located at least 9 nucleotides distance from the free end of the second segment;
wherein a free end of the first segment of HPA1 is capable of ligating to a first end of a sample ssDNA, and a free end of the first segment of HPA2 is capable of ligating to a second end of a sample ssDNA.

2. The composition of claim 1, wherein the HPA1 and the HPA2 consist of:
i) a first segment comprising at least one primer-specific or probe-specific sequence;
ii) a second segment comprising a sequence substantially complementary to the first segment; and
iii) a loop connecting the first segment and the second segment;
wherein a free end of the second segment comprises an overhang of at least one residue,
wherein the second segment comprises a cleavable moiety located at least 9 nucleotides distance from the free end of the second segment;
wherein a free end of the first segment of HPA1 is capable of ligating to a first end of a sample ssDNA, and a free end of the first segment of HPA2 is capable of ligating to the other segment of the sample ssDNA.

3. The composition of claim 1 or 2, wherein the free end of the second segment of the HPA1, the free end of the second segment of the HPA2, or the free end of the second segment of the HPA1 and the free end of the second segment of the HPA2 comprise a blocking group that prevents its ligation by a ligase and/or extension by a polymerase.

4. The composition of any one of claims 1 to 3, wherein the primer-specific and/or probe-specific sites are at least partially complementary or corresponding to sequences of primers and probes selected from: amplification primers, sequencing primers, detection primers, detection probes, hybridization probes, anchor probes, linker probes, capture probes or combination thereof.

5. The composition of any one of claims 1 to 4, wherein the first segment and the second segment form a double-stranded stem structure with at least 1, 2, 3, or more mismatches.

6. The composition of any one of claims 1 to 5, wherein the double-stranded stem structure comprises 3 or more nucleotide base pairs.

7. The composition of any one of claims 1 to 6, wherein the loop comprises 1 or more residues selected from: a nucleotide, a modified nucleotide and a non-nucleotide linker or moiety.

8. The composition of any one of claims 1 to 7, wherein the cleavable residue or moiety is selected from: a RNA, a deoxyuridine (dU), a deoxyinosine (dI); an internucleotide disulfide (S—S) linker and an internucleotide, bridging or non-bridging phosphorothioate (PS) linkage.

9. The composition of any one of claims 1 to 8, wherein the overhang comprises three or more randomized nucleotide residues (N) or defined nucleic acid and/or modified nucleic acid residues or combinations thereof, allowing simultaneous ligation with any sample ssDNA regardless of its end sequences.

10. The composition of claim 9, wherein the ligation is target-specific.

11. The composition of claim 10, wherein the adaptor overhang comprises a sequence that is substantially complementary to a sequence of a sample ssDNA end.

12. The composition of any one of claims 1 to 11, wherein the overhang comprises from 3 to 12 randomized residues selected from: nucleotide residues, modified nucleotide residues, or a combination thereof.

13. The composition of any one of claims 1 to 12, wherein the modified nucleotide residues are selected from the list consisting of Locked nucleic acids, 2′-OMethyl, 2′-Fluoro, 2-Amino-dA, 5-Methyl-dC, C-5 propynyl-C, and C-5 propynyl-U.

14. The composition of any one of claims 1 to 13, wherein the free end of HPA1 is selected from: 5′-hydroxyl (5′-OH), 5′-phosphate (5′-p), 3′-hydroxyl (3′-OH), 3′-phosphate (3′-p) or combination thereof.

15. The composition of any one of claims 1 to 14, wherein the free end of HPA2 is selected from: 5′-OH, 5′-p, 3′-OH, 3′-p or combination thereof.

16. The composition of any one of claims 1 to 15, wherein the HPA1 or the HPA2 is a 3′-HPA that can be ligated to the 3′-end of a sample ssDNA.

17. The composition of any one of claims 1 to 15, wherein the HPA1 or the HPA2 is a 5′-HPA that can be ligated to the 5′-end of a sample ssDNA.

18. The composition of any one of claims 1 to 17, further comprising a blocking oligonucleotide (BON) comprising:
i) a free end that enables ligation of the BON with any HPA1 remaining unligated after the the ligation of the HPA1 to the ssDNA to reduce ligation between the HPA1 and the HPA2; and
ii) a second end.

19. The composition of claim 18, wherein the second end of the BON comprises a blocking group that disallows its ligation and/or extension by a polymerase.

20. The composition of claim 18 or 19, wherein the BON comprises a structure selected from: single-stranded, double-stranded, hairpin, or combination thereof.

21. The composition of any one of claims 18 to 20, wherein the BON comprises a nucleotide selected from: DNA, RNA; a randomized nucleotide residue (N); a defined nucleic acid residue; a modified nucleic acid residue; or a combination thereof.

22. The composition of claim 21, wherein the BON comprises from 1 to 12 randomized nucleotide residues.

23. The composition of any one of claims 18 to 22, wherein the free end of the BON or the second end of the BON comprises sequences selected from 4 to 6 random nucleotides.

24. The composition of any one of claims 18 to 23, wherein the free end of the BON or the second end of the BON are selected from the group consisting of: a 5′-OH, 5′-p, 3′-OH, and combinations thereof.

25. The composition of any one of claims 3 to 24, wherein the blocking group is selected from: 5′-OH, 5′-amino, 5′-O-methyl and 5′-biotin linker (5′-end blocking groups); and 3′-p, dideoxynucleoside (e.g., 3′-ddC), 3-inverted dT (idT), 3′-C3 spacer, 3′-amino, and 3′-biotin linker (3′-end blocking groups).

26. A method for preparing a plurality of primer extension products for a plurality of sample ssDNAs, comprising:
a) ligating the HPA1 of claim 1 to the first end of a plurality of sample ssDNAs to produce a mixture comprising a plurality of HPA1-ssDNA or ssDNA-HPA1 ligation products and unligated HPA1;
b) ligating the HPA2 of claim 1 to the second end of the HPA1-ssDNA ligation products to produce 5′-HPA-ssDNA-3′-HPA (5′-HPA1-ssDNA-HPA2-3′ or 5′-HPA2-ssDNA-HPA1-3′) ligation products, wherein the sample ssDNA is positioned between the HPA1 and HPA2;
c) cleaving the of the HPA(s) to produce cleaved 5′-HPA-DNA-3′-HPA ligation products, wherein the cleaving converts the HPAs to opened forms that are more accessible for hybridization with primer(s) and/or probe(s); and
d) hybridizing a first primer comprising a sequence at least partially complementary to the 3′-HPA(s) segment of the cleaved 5′-HPA-ssDNA-3′-HPA products; and extending the primer with a polymerase to produce a plurality of the first primer extension products.

27. The method of claim 26, further comprising (e) hybridizing a second primer comprising a sequence at least partially complementary to the 3′-end of the first primer extension products comprising a sequence complementary to the 5′-HPA segment, and extending the primer with a polymerase to produce a plurality of the second primer extension products comprising a sequencing library.

28. The method of claim 26 or 27, wherein the ligating of the HPA1 in step (a) occurs before the ligating of HPA2 in step (b).

29. The method of any one of claims 26 to 28, wherein the ligating of the HPA1 in step (a) and the ligating of the HPA2 in step (b) occur simultaneously.

30. The method of any one of claims 26 to 29, further comprising step (f) ligating any HPA1 remaining unligated after step (a) with the BON of claim 18 to produce HPA1-BON ligation product(s) that prevent its ligation to HPA2 (adapter dimers formation) in a downstream ligation reaction.

31. The method of any one of claims 26 to 30, further comprising removing components of upstream reactions that may inhibit downstream primer extension reactions.

32. The method of claim 31, wherein the removing is performed using SPRIselect beads and reagents.

33. The method of any one of claims 26 to 32, further comprising amplifying the sequencing library to produce an amplified sequencing library.

34. The method of any one of claims 26 to 33, further comprising performing a post-amplification clean-up to purify the amplified sequencing library from non-extended primers and other components of extension and/or amplification reactions.

35. The method of any one of claims 26 to 34, wherein the sample ssDNAs are naturally occurring and/or synthetic DNA molecules selected from: single-stranded DNAs; fragmented single-stranded DNAs; denatured double-stranded DNAs; denatured fragmented double-stranded DNAs.

36. The method of any one of claims 26 to 35, wherein the sample ssDNAs are short ssDNA fragments of 120 or fewer nucleotides in length.

37. The method of any one of claims 26 to 35, wherein the sample ssDNAs are ultrashort ssDNA fragments in the range of 20 to 80 nucleotides in length.

38. The method of any one of claims 26 to 35, wherein the sample ssDNAs are in the range of 18 to 50 nucleotides in length.

39. The method of any one of claims 26 to 38, wherein the sample ssDNAs are selected from: circulating tumor DNA, circulating microbial DNA, circulating bacterial DNA, circulating viral DNA, circulating mitochondrial DNA, circulating genomic DNA, circulating cell-free DNA (cfDNA) from a biofluid (liquid biopsy); DNA from formalin-fixed, paraffin-embedded (FFPE) tissue samples; highly degraded DNA from ancient organisms or forensic biological samples.

40. The method of claim 39, wherein the biofluid is selected from: whole blood, plasma, serum, saliva, and urine.

41. The method of any one of claims 26 to 40, wherein the sample ssDNA comprises isolated total nucleic acids including both DNA and RNA.

42. The method of any one of claims 26 to 40, wherein the sample ssDNA comprises isolated total DNA.

43. The method of any one of claims 26 to 40, wherein the sample ssDNA comprises isolated target DNAs.

44. The method of any one of claims 26 to 43, wherein the target ssDNAs are isolated by hybridization with target-specific oligonucleotide probes.

45. The method of claim 44, wherein the hybridization is performed either in solution followed by attachment of target-probe complexes to a solid support, or on a solid phase comprising target-specific probes immobilized on a solid support.

46. The method of any one of claims 43 to 45, wherein the target ssDNAs are captured directly from a biofluid or a lysed biofluid.

47. The method of any one of claims 26 to 46, wherein the plurality of sample ssDNAs comprise DNA ends selected from 5′-p, 3′-OH, 5′-OH and 3′-p or combinations thereof.

48. The method of any one of claims 26 to 47, further comprising chemically or enzymatically treating the sample ssDNAs.

49. The method of claim 48, wherein chemically or enzymatically treating the sample ssDNAs comprises a bisulfite treatment protocol that can convert unmethylated cytosine (C) to deoxyuridine (dU).

50. The method of claim 48, wherein chemically or enzymatically treating the sample ssDNAs comprises repairing the sample ssDNA to convert 5′-OH and/or 3′-p ends to 5′-p and 3′-OH forms.

51. The method of claim 50, wherein the repairing is performed by a polynucleotide kinase.

52. The method of any one of claims 26 to 47, wherein the ligating is performed without repair of ends of the sample ssDNA.

53. The method of any one of claims 26 to 52, wherein the ssDNAs are chemically or enzymatically treated prior to the ligating of HPA1 in step (a) or the ligating of HPA2 in step (b).

54. The method of any one of claims 26 to 52, wherein the ssDNAs are chemically or enzymatically treated simultaneously with the ligating of HPA1 in step (a) or the ligating of HPA2 in step (b).

55. The method of any one of claims 26 to 54, wherein the HPA1 is a 3′-HPA and the HPA2 is a 5′-HPA.

56. The method of any one of claims 26 to 54, wherein the HPA1 is the 5′-HPA and the HPA2 is the 3′-HPA.

57. The method of claim 55 or 56, wherein the 3′-HPA comprises a 5′-p end and its 3′-overhang comprises a 3′-end blocking group.

58. The method of claim 55 or 56, wherein the 5′-HPA comprises a 3′-OH end and a 5′-end overhang comprising a 5′-OH or a 5′-end blocking group.

59. The methods of claim 57, wherein the remaining unligated 3′-HPA is blocked by ligating with a 3′-blocking oligonucleotide (3′-BON) comprising a 3′-OH and a 5′-OH or 5′-end-blocking group.

60. The method of claim 58, wherein the remaining unligated 5′-HPA is blocked by ligating with a 5′-blocking oligonucleotide (5′-BON) comprising a 5′-p end and a 3′-end-blocking group.

61. The method of any one of claims 26 to 60, wherein the HPA2 is taken in molar excess over the HPA1.

62. The method of any one of claims 30 to 61, wherein the BONs are taken in molar excess over the HPA1 during the blocking step.

63. The method of any one of claims 26 to 62, wherein the ligating is splint-dependent ligation between the sample ssDNAs, the HPA1, the HPA2 and/or the BON and a ligation step performed by a ligase.

64. The method of claim 63, wherein the ligase is selected from: Salt-T4@ DNA Ligase, T4 DNA ligase, T3 DNA ligase, T7 DNA ligase, PBCV1 DNA ligase, E. coli DNA ligase, human DNA ligase III, and SplintR® Ligase.

65. The method of claim 64, wherein the ligating by T4 DNA ligase is performed in the presence of ATP at a concentration from about 50 to about 100 μM.

66. The method of any one of claims 26 to 65, wherein the HPA1 or the HPA2 comprise one or more RNA residues that can be cleaved by an RNA cleaving agent selected from: a ribonuclease, a ribozyme, a deoxyribozyme, basic buffer solutions, alkaline solutions, divalent or multivalent metal cations, or combinations thereof.

67. The method of claim 66, wherein the ribonuclease comprises RNase H.

68. The method of claim 66, wherein the use of a RNA cleaving agent for the HPA cleavage simultaneously cleaves any RNA present in the sample ssDNA to prevent the incorporation of RNA sequences into the ssDNA sequencing library.

69. The method of claim 66, wherein the RNA cleaving agent is using oligonucleotides comprising sequence-specific or randomized nucleotides to guide a cleavage of RNA molecules incorporated into the ssDNA sequencing library.

70. The method of any one of claims 33 to 69, wherein the amplification is performed by PCR using a thermostable DNA polymerase.

71. The method of claim 70, wherein the thermostable DNA polymerase is selected from: LongAmp HotStart Taq DNA polymerase, KAPA HiFi HotStart DNA polymerase, KAPA HiFi HotStart Uracil+DNA polymerase; Pfu Turbo Cx HotStart DNA polymerase.

72. A method for preparing a sequencing library from a plurality of sample ssDNAs, comprising:
a) ligating a 5′-hairpin adapter (5′-HPA) to the 5′-ends of the plurality of sample single-stranded DNA (sample ssDNAs) to produce a mixture comprising a plurality of 5′-HPA-ssDNA ligation products, wherein the 5′-HPA is comprised of a plurality of residues selected from: nucleic acid residues (DNA and RNA), modified nucleic acid residues, non-nucleotide residues or a combination thereof, wherein the 5′-HPA comprises:
i) a first segment comprising at least one primer primer-specific sequence;
ii) a second segment comprising a sequence substantially complementary to the first segment,
iii) a loop connecting the first segment and the second segment;
iv) free 3′-end and 5′-end which are not connected to the loop;
wherein the free 3′ end of the first segment comprises 3′-OH; and the 5′ end of the second segment comprises an overhang of 5 or 6 randomized nucleotide residues and 5′-OH; and
wherein the second segment comprises 2 or more RNA residues in its stem region located at least 9 nucleotides distance from the free 5′ end of the second segment; and
b) ligating the 3′-hairpin adapter (3′-HPA) to the 3′ ends of the 5′-HPA-ssDNA ligation products to produce 5′-HPA-ssDNA-3′-HPA ligation products; wherein the 3′-HPA is comprised of a plurality of residues selected from: nucleic acid residues (DNA and RNA), modified nucleic acid residues, non-nucleotide residues or a combination thereof; wherein the 3′-HPA comprises:
i) a first segment comprising at least one primer primer-specific sequence;
ii) a second segment comprising a sequence substantially complementary to the first segment,
iii) a loop connecting the first segment and the second segment;
iv) free 5′-end and 3′-end which are not connected to the loop;
wherein the free 5′ end of the first segment comprises 5′-p; and 3′ end of the second segment comprises an overhang of 5 or 6 randomized nucleotide residues and 3′-blocking group that prevents its ligation and/or extension by a polymerase; and
wherein the second segment comprises 2 or more RNA residues in its stem region; and
c) cleaving the RNA residues in both 5′-HPAs and 3′-HPAs by treatment with an RNase H to produce cleaved 5′-HPA-ssDNA-3′-HPA ligation products, wherein the cleaving makes the first segments of 5′-HPA and 3′-HPA more accessible for hybridization with primers;
d) performing a pre-PCR clean-up of the cleaved 5′-HPA-ssDNA-3′-HPA ligation products to deplete or remove other components and side products of upstream ligation and cleavage reactions (including adapter dimers 5′-HPA-3′-HPA and inhibitors of the PCR) before the PCR amplification;
e) amplifying the cleaved 5′-HPA-ssDNA-3′-HPA ligation products by a PCR using a pair of the first and the second sequencing primers to produce (amplified) sequencing library, wherein:
i) the first sequencing primer is at least partially complementary at its 3′ end to the 3′-HPA segment of the cleaved 5′-HPA-ssDNA-3′-HPA ligation products; and
ii) the second sequencing primer is at least partially corresponding at its 3′ end to the 5′-HPA segments of the cleaved 5′-HPA-ssDNA-3′-HPA ligation products;
f) performing a post-amplification clean-up to purify the (amplified) sequencing library from non-extended primers and other components of extension and/or amplification reactions to produce purified sequencing libraries that are ready for sequencing.

73. The method of claim 72, wherein the ligating of 3′-HPA (but not 5′-HPA) is performed in the presence of polynucleotide kinase (PNK).

74. The method of any one of claims 26 to 73, wherein the HPA1, the HPA2 and the sequencing primers are compatible with current high-throughput, sequencing technologies selected from:
second-generation or next-generation sequencing (NGS) and third-generation or direct, single-molecule sequencing.

75. The method of claim 74, selected from sequencing methods provided by sequencing-by-synthesis, single-molecule real time sequencing, and nanopore sequencing.

76. The method of any one of claim 26 to 74, wherein the primers are sequencing primers comprising molecular codes selected from: bar-codes, sequencing indexes, unique molecular identifiers (UMI or UID) or combination thereof.

77. A kit for preparing the sample ssDNA sequencing library comprising a 5′-HPA, a 3′-HPA, a ligase, an RNA cleaving agent, buffers, and optional components selected from: PNK, a polymerase, a 5′-BON, 3′-BON, and clean-up beads or combinations thereof.
